(19) United States
(12) Patent Application Publication
(10) Pub. No.: US 2024/0060995 A1
(43) Pub. Date: Feb. 22, 2024

(54) INNATE IMMUNE PROTEINS AS BIOMARKERS FOR TRAUMATIC BRAIN INJURY IN ADULT AND PEDIATRIC PATIENTS

(71) Applicant: University of Miami, Miami, FL (US)

(72) Inventors: Juan Pablo de Rivero Vaccari, Miami, FL (US); Robert W. Keane, Miami, FL (US); W. Dalton Dietrich, Miami, FL (US); Jennifer Christine Munoz-Pareja, Miami, FL (US); Nathan H. Johnson, Miami, FL (US); Jon Perez-Barcena, Miami, FL (US)

(21) Appl. No.: 18/306,956

(22) Filed: Apr. 25, 2023

Related U.S. Application Data
(63) Continuation-in-part of application No. 16/986,904, filed on Aug. 6, 2020, now abandoned.
Application No. 16/986,904 is a continuation of application No. 15/214,868, filed on Jul. 20, 2016, now abandoned.
Application No. 15/214,868 is a continuation of application No. 14/376,383, filed on Aug. 1, 2014, now abandoned.
Application No. 14/376,383 is a continuation-in-part of application No. 17/921,600, filed on Oct. 26, 2022.
(60) Provisional application No. 63/334,218, filed on Apr. 25, 2022.
(60) Provisional application No. 61/595,254, filed on Feb. 6, 2012.
(60) Provisional application No. 63/062,622, filed on Aug. 7, 2020.
(60) Provisional application No. 63/016,033, filed on Apr. 27, 2020.

(51) Int. Cl. G01N 33/68; A61F 7/00

(52) U.S. Cl. CPC G01N 33/6896; A61F 7/00; G01N 2800/28; G01N 2800/52; G01N 2800/7095

(57) ABSTRACT

An array of inflammatory cytokines are useful as a biomarker of traumatic brain injury, including inflammasome proteins. Biomarker cut-off points, ROC, and other characteristics have been determined.

### CROSS-REFERENCE TO RELATED APPLICATIONS

This application claims the benefit of U.S. Provisional Patent Application, Ser. No. 63/334,218, filed Apr. 25, 2022.

This application is a continuation-in-part of pending U.S. patent application Ser. No. 16/986,904, filed Aug. 6, 2020, which is a continuation of U.S. Potent Application, Ser. No. 15/214,868, filed Jul. 20, 2016, now abandoned, which is a continuation of U.S. patent application Ser. No. 14/376,383, filed Aug. 1, 2014, now abandoned, which is a national stage entry (§ 371 filing) from International Potent Application No. PCT/US2013/024941, filed Feb. 6, 2013, claiming the benefit of U.S. Provisional Patent Application, Ser. No. 61/595,254, filed Feb. 6, 2012.

This application is also a continuation-in-part of pending U.S. Potent Application, Ser. No. 17/921,600, filed Oct. 26, 2022, which is a national stage entry (371 filing) from International Potent Application No. PCT/US2021/029419, filed Apr. 27, 2021, claiming the benefit of U.S. Provisional Potent Application, Ser. No. 63/062,622, filed Aug. 7, 2020, and of U.S. Provisional Patent Application, Ser. No. 63/016,033, filed Apr. 27, 2020 Each of the above patent applications from which this application claims priority or the benefit of the filing date is hereby incorporated by reference in its entirety.

### FIELD OF THE INVENTION

The present invention relates generally to the fields of neurology, immunology, and diagnostics. In particular, the present invention relates to the identification of biomarkers in biological samples which can predict the severity of neuronal injury, such as spinal cord and traumatic brain injury, in patients. The identified biomarkers may also be used in determining prognosis, directing therapeutic and rehabilitation efforts, and monitoring response to treatment for patients with a central nervous system injury.

### STATEMENT REGARDING SEQUENCE LISTING

The Sequence Listing associated with this application is provided in ST.26 (XML) format in lieu of a paper copy, and is hereby incorporated by reference into the specification. The name of the XML file containing the Sequence Listing is 9842-8701US_SeqList_ST26.xml. The XML file is ˜53.8 KB, and was created on Apr. 25, 2023, and is being submitted electronically via EFS-Web.

### BACKGROUND OF THE INVENTION

Nucleotide-binding oligomerization domain (NOD)-containing protein-like receptors (NLRs) are a recently discovered class of innate immune receptors that play a crucial role in initiating inflammatory responses following tissue injury in the central nervous system (CNS) (Abulafia et al., 2009, Silverman et al., 2009). Previous work shows that NLRP1 (also known as NAcht leucine-rich-repeat protein 1 (NALP-1)) forms an inflammasome complex comprising NLRP1, the adaptor protein apoptosis-associated speck-like protein containing a caspase recruitment domain (ASC) and the caspase-1 enzyme that orchestrate the early inflammatory processes after spinal cord injury (SCI) and traumatic brain injury (TBI) via IL- I 13 activation (de Rivero Vaccari et al., 2008; 2009). The formation of inflammasomes is induced by physical damage to the plasma membrane, and by certain endogenous ligands referred to as danger associated molecular patterns (DAMPs) or exogenous ligands known as pathogen associated molecular patterns (PAMPs) (Bianchi, 2007, Wakefield et al., 2010). However, the full IL-10 response also depends on the activation of Toll-like receptors (TLRs) and/or purinergic ATP-gated receptors, which induce the transcription of pro-IL-10.

Hyperinflammatory responses associated with tissue damage can promote pathogenesis of SCI and TBI via overproduction of IL-10 and other potentially neurotoxic products. Inflammasome-mediated IL-1β overproduction is involved in the pathogenesis of type 2 diabetes, liver damage and muscular dystrophy (Kufer and Sansonetti, 2011). Moreover, increasing genetic evidence suggests that inflammasome activation could also drive adaptive immunity in types of dermatitis, skin related allergies and asthma (Kufer and Sansonetti, 2011). In addition, inflammasome components may be secreted into the extracellular milieu via a mechanism involving the exosome pathway (Bianchi, 2007). The inflammasome therefore has a complex connection with the control of adaptive immune responses that has become the subject of intense investigation. Whether inflammasomes are associated with tissue destructive inflammatory processes after SCI and TBI in humans has not been investigated.

Traumatic brain injury (TBI) has a complex, biphasic pathology that represents a significant public health concern in the United States and throughout the world. TBI affects an estimated 1.5 million people each year and causes one-third of injury-related deaths. Approximately 5.3 million Americans are living today with a permanent TBI-related disability representing an annual economic impact in excess of $56 billion. Predicting the severity and outcome of TBI and well as SCI is difficult, given the lack of objective, laboratory-based biomarkers. Currently, the Glasgow Coma Scale (GCS) score (Teasdale et al., 1974) is the best available clinical predictor of injury severity; however, its value is limited in patients undergoing pharmacological paralysis for intubation, as a motor score cannot be obtained (Brain Trauma Foundation, American Association of Neurological Surgeons, 2000). Predicting outcome is further complicated by the heterogeneity of pathology in patients with a similar GCS score. Therefore, the identification of diagnostic and prognostic biomarkers that directly reflect injury to CNS cells is imperative. Such biomarkers of TBI and SCI will enable clinicians to assess the degree of damage to the brain or spinal cord, relay prognostic information to the patient's family members, and target acute and chronic treatments to specific CNS damage mechanisms. Therefore, an early, accurate diagnostic test designed to target neuroprotective strategies would be a most desirable prognostic tool.

Although significant progress has been made regarding the verification and testing of various biomarkers after stroke and TBI, limited data are available regarding what biomarkers are appropriate for SCI. The biomarkers S-10013, neuron-specific enolase, neurofilament light chain and glial fibrillary acidic protein are significantly increased in cases of SCI in experimental animal studies (Skouen et al., 1999, Ma et al., 2001, Nagy et al., 2002, Cornefijord et al., 2004, Loy et al., 2005, Cao et al., 2008, Pouw et al., 2009). Although some biomarkers show promising results, these do not yet provide a sensitive prognostic tool. Quantitative standards for determining the extent of SCI and TBI during the acute phase must be developed and validated.

Damage-associated proteins released upon initial brain injury exacerbate the neuroinflammatory response during the chronic secondary injury period, resulting in persistent inflammation and the chronic activation of the innate immune system. Primary injury involves the release of damage associated molecular patterns (DAMPS) and pathogen associated molecular patterns (PAMPS) from injured tissue. PAMPs and DAMPs are recognized by toll like receptors (TLR) which along with TBI-induced cellular potassium efflux, increased intracellular calcium, subsequent mitochondrial dysfunction, and excitotoxicity result in activation and formation of the inflammasome. Although the levels of DAMPs and PAMPs have been shown to gradually decrease over the first week after injury, activation of the innate immune response results in formation of the inflammasome.

The inflammasome is a multi-protein complex that allows for the activation of caspase-1 using a scaffolding protein known as apoptosis-associated speck-like protein containing a caspase recruiting domain (ASC.) Oligomerization of ASC forms an ASC speck that binds to caspase-1, resulting in formation of the inflammasome complex and downstream activation of the pro-inflammatory cytokines interleukin (IL)-18 and IL-10, allowing for the cleavage and release of the inflammatory cytokines interleukin (IL)-10 and IL-18, as well as the formation of a gasdermin-D pore as part of the programmed cell death mechanism of pyroptosis.

Inflammasome formation can be triggered through numerous vectors and has been shown to be activated after TBI in rodents and humans. Increased levels of inflammasome proteins during the acute phase after TBI are associated with worsened functional outcomes. Chronic inflammatory activity is often seen months to years after injury, resulting in a secondary injury from chronically activated microglia and their subsequent release of inflammatory cytokines.

Our own previous studies revealed that the level of inflammasome proteins in biological fluids can be used as biomarkers for determination of TBI functional outcomes. What is needed is an accurate method of determining injury severity and which is predictive of pathological outcomes

A new approach for evaluating the primary cord and brain damage in the acute phase is the assessment of biomarkers in the cerebrospinal fluid (CSF). Since CSF surrounds the spinal cord and brain, damage to the cord or brain may lead to the release of proteins and molecules from central nervous system cells into the CSF that may serve as biomarkers for SCI and TBI in the CSF. Several studies have been conducted concerning S-10013, neuron-specific enolase, neurofilament light chain, and glial fibrillary acidic protein (GF AP) in CSF and serum of animal models of SCI (Pouw et al., 2009). However, only one study has investigated neurofilament protein and GF AP in CSF after SCI in humans (Guez et al., 2003). Thus, there is a need in the art to identify biomarkers of neuronal damage following central nervous system injury in humans that can be used to ascertain the severity of the injury and facilitate the selection of an appropriate therapeutic strategy to maximize recovery.

### SUMMARY OF THE INVENTION

The present invention is based, in part, on the discovery that NLRP1 (NALP-1) inflammasome components are secreted into the cerebrospinal fluid (CSF) acutely after SCI and traumatic brain injury (TBI) in humans. Elevated inflammasome protein levels in the CSF following central nervous system (CNS) injury represent the degree of neuroinflammation in CNS tissue and reflect the extent of inflammatory-induced damage. The CSF levels of inflammasome protein following injury correlate with the degree of functional recovery in patients and thus, can be used as acute biomarkers to predict patient prognosis and direct therapeutic interventions. Accordingly, the present invention provides a method of assessing the severity of a CNS injury in a patient, including an accurate and predictable determination and diagnosis of traumatic brain injury (TBI).

In one embodiment, the invention provides a method of evaluating a patient suspected of having a CNS injury comprising providing a biological sample from a patient presenting with clinical symptoms consistent with a CNS injury, measuring the level of at least one inflammasome protein in the biological sample, determining the presence or absence of a protein signature associated with a CNS injury or a more severe CNS injury, such as traumatic brain injury (TBI), wherein the protein signature comprises an elevated level of said at least one inflammasome protein, and selecting patients exhibiting the presence of the protein signature as having a CNS injury or a more severe CNS injury. Tn certain embodiments, said one or more inflammasome proteins are NLRP1 (NALP-1), ASC, or caspase-1. The diagnostic methods of the invention may further comprise administering a neuroprotective treatment to the patient based on the measured level of one or more inflammasome proteins, and following changes in the level of one or more inflammasome proteins as a mechanism to monitor response to treatment.

In some embodiments, the levels of one or more inflammasome proteins in the patient's sample can be used to prepare an inflammasome protein profile associated with CNS injury. The levels of inflammasome proteins in the profile may be determined relative to levels of the proteins in control samples or pre-determined reference values or ranges of reference values. The inflammasome protein profiles are, in some embodiments, indicative of the presence or severity of CNS injury in a patient. When such protein profiles are prepared from samples obtained from patients following administration of a neuroprotective treatment, the inflammasome protein profiles are indicative of therapeutic efficacy of the neuroprotective treatment in the patient. In particular, the subject invention includes the discovery of cut-off values for certain immune proteins or inflammasome proteins which can determine whether the CNS injury is traumatic brain injury (TBI).

The present invention also provides a method of determining a prognosis for a patient with a central nervous system injury. In one embodiment, the method comprises providing a biological sample, such as cerebrospinal fluid, obtained from the patient shortly after injury (e.g., within a week of injury), and measuring the level of at least one inflammasome protein in the biological sample to prepare an inflammasome protein profile, wherein the inflammasome protein profile is indicative of the prognosis of the patient. In particular embodiments, an elevated level of at least one inflammasome protein relative to a pre-determined reference value or range of reference values is indicative of a poorer prognosis or unfavorable patient outcome. For example, elevated inflammasome protein levels are predictive of the patient having a Glasgow Outcome Scale (GOS) score of 1 to 3 upon follow-up assessment. In other embodiments, a reduced level of at least one inflammasome protein relative to a pre-determined reference value or range of reference values is predictive of a favorable patient outcome (e.g., GOS score of 4 or 5 upon follow-up assessment). In certain embodiments, the method provides a prognosis for a patient with a spinal cord or traumatic brain injury.

The present invention also includes kits for preparing an inflammasome protein profile associated with CNS injury. In one embodiment, the kit comprises a labeled-binding partner, such as labeled-antibody or fragment thereof, that specifically binds to one or more inflammasome proteins, wherein said one or more inflammasome proteins are selected from the group consisting of NLRP1, ASC, caspase-1, and combinations thereof.

We analyzed blood serum from TBI patients and respective controls utilizing Simple Plex inflammasome and V-PLEX inflammatory cytokine assays. We performed statistical analyses to determine which proteins were significantly elevated in TBI individuals. Next, we determined the receiver operating characteristics (ROC) to obtain the area under the curve (AUC) to determine the potential fit as a biomarker. Potential biomarkers were then compared to documented patient Glasgow coma scale scores via a correlation matrix and a multivariate linear regression to determine how respective biomarkers related to the injury severity and pathological outcome.

Inflammasome proteins and inflammatory cytokines were elevated after TBI, and that apoptosis-associated speck like protein containing a caspase recruitment domain (ASC), interleukin (IL)-18, tumor necrosis factor (TNF)-α, IL-4 and IL-6 were the most promising biomarkers. Additionally, levels of these proteins correlated with known clinical indicators of pathological outcome, such as Glasgow coma scale (GCS). Our results show that inflammatory cytokines and inflammasome proteins are promising biomarkers for determining pathological outcomes after TBI. Additionally, levels of biomarkers could potentially be utilized to determine a patient's injury severity and subsequent pathological outcome.

These findings show the potential for the use of inflammatory associated proteins as biomarkers and offer an additional method of TBI patient assessment.

In addition, protective or therapeutic treatment of traumatic brain injury (TBI) can be administered to a patient suffering from TBI, as determined by the disclosed method. Specifically, a small molecule drug or large molecule, such as a peptide, protein, antibody, or the like, binding to one or more of the specific immune protein or inflammasome biomarkers identified herein, presenting at levels meeting the cut-off value, or within 20% of the cut-off value for that biomarker, can be administered to the patient in an effective amount such that the immune protein or inflammasome biomarker, or its cascade production, is inactivated or disabled.

### BRIEF DESCRIPTION OF DRAWINGS

FIG. 1. NLRP1, ASC, and Caspase-1 are biomarkers that predict outcome after SCI. CSF samples were immunoblotted with antibodies against NLRP1, caspase-1 and ASC. CSF samples from uninjured patients were used as controls. Immunoblot analysis of 6 different cases of patients with SCI indicates that patients (2, 3 and 4) who express low levels of caspase-1 acutely after SCI have a better prognosis than subjects (1, 5 and 6) who have elevated levels of this protein in CSF.

FIG. 2. NLRP1 inflammasome proteins are expressed in cells of the CNS. Spinal cord sections were obtained from decedents that had injury to the spinal cord. Immunohistochemical analysis combined with light microscopy indicates that NLRP1 is expressed in neurons of the ventral horn (black arrows) myelinated axons (black arrow heads) and oligodendrocytes (yellow arrows) (top panel).

Caspase-1 is expressed in swollen axons (spheroids, blue arrows), motor neurons (black arrows) and in oligodendrocytes (yellow arrows) (central panel). ASC is present in neurons in the ventral horn (black arrows), white matter oligodendrocytes (yellow arrow) and macrophages/microglia (blue arrow heads) (bottom panel).

FIG. 3. Scatter plots of expression of inflammasome proteins in controls and patients with TBI. Samples were immunoblotted for ASC (A), caspase-1 (B), and NALP-1 (C). The p values in the upper left corner represent results of a Mann-Whitney U-test. Densitometric analysis revealed a significant increase in expression of ASC, caspase-1 (p20), and NALP-1 in the CSF of patients with TBI compared with nontrauma controls. Solid lines denote mean values for each group. Different shapes correspond to patient outcomes at 5 months postinjury. Representative immunoblots are shown. Samples were run on the same gel but were noncontiguous. N=the number of TBI samples analyzed; ★=GOS Score 5; ▪=GOS Score 4; ⊙=GOS Score 3; ∇=GOS Score 1.

FIG. 4. Box plots of expression of inflammasome proteins sorted by outcome category. The ends of the whiskers represent the lowest datum within 1.5 interquartile range of the lower quartile and the highest datum within 1.5 interquartile range of the upper quartile. The asterisks represent the outliers. Mann-Whitney U-tests indicate higher expression of ASC (A), caspase-1 (p20) (B), and NALP-1 (C) are significantly associated with an unfavorable outcome 5 months after injury (p<0.0001). Representative immunoblots for each protein are shown. Samples were run on the same gel but were noncontiguous.

FIG. 5. Scatter plots and estimated linear regression of ASC (A), caspase-1 (p20) (B), and NALP-1 (C) expression in the CSF with GOS score. Probability values of the linear regression are shown in the top left of each graph. Expression of each protein correlated significantly with GOS score at 5 months post-injury. The p values on the x axis represent post hoc comparisons of a Kruskal-Wallis test. Representative immunoblots are shown. Samples were run on the same gel but were noncontiguous.

FIG. 6. Caspase-1 levels in CSF one, two, and three days following TBI in pediatric patients receiving hypothermia treatment or no treatment (normothermia).

FIG. 7: Inflammatory cytokines and Inflammasome Proteins are Elevated after TBI. Simple Plex Assay and MSD-VPLEX Inflammatory Panel of blood serum from TBI patients and age matched controls. Data were analyzed utilizing a two-tailed Mann-Whitney nonparametric test. Inflammatory cytokines and inflammasome proteins that showed statistically significant increase after TBI were plotted. Box and whisker plots show mean, quartiles, and outliers for each inflammatory protein of interest with respective p values listed above. Dots correspond to data points outside the 5th and 95th percent confidence interval. Results showed that A) Caspase-1: N: Control: 31, TBI: 78; B) ASC: N: Control: 28, TBI: 91; C) IL-18: N: Control: 31, TBI: 90; D) TNF-α: N: Control: 21, TBI: 51; E) IL-6: N: Control: 21, TBI: 46; and F) IL-4: N: Control: 20, TBI: 50; G) IL-10: N: Control: 19, TBI: 41; H) IL-8: N: Control: 12, TBI: 52 were all significantly elevated in TBI patients when compared to controls. 1) IL-2: N: Control: 9, TBI: 19 was significantly decreased in TBI patients when compared to controls.

FIG. 8: ROC of Inflammatory Biomarkers. ROC and AUC were calculated for each inflammatory cytokine and inflammasome proteins that were significantly different when comparing healthy uninjured controls and TBI patients. A) Caspase-1: N: Control: 31, TBI: 78; B) ASC: N: Control: 28, TBI: 91; C) IL-18: N: Control: 31, TBI: 90; D) TNF-α: N: Control: 21, TBI: 51; E) IL-6: N: Control: 21, TBI: 46; and F) IL-4: N: Control: 20, TBI: 50; G) IL-10: N: Control: 19, TBI: 41; H) IL-8: N: Control: 12, TBI: 52; 1) IL-2: N: Control: 9, TBI: 19.

FIG. 9: ROC Comparison among Inflammatory Biomarkers. A) Correlation matrix using a Pearson correlation among inflammatory Biomarkers. B) p-values of significance for the comparison among the inflammatory proteins analyzed: caspase-1, ASC, IL-18, TNF-α, IL-6, IL-4, IL-10 and IL-8.

FIG. 10: Inflammatory biomarkers as predictors of TBI severity. A) Box and whisker plots showing the protein levels in pg/mL of IL-13 as a biomarker of injury severity. Box and whiskers are shown for the 5th and 95th percentile. Dots correspond to data points outside the 5th and 95th percent confidence interval. B) ROC curves indicate the AUC (sensitivity vs. 1-specificity) for IL-13. Mild: N=14, Moderate to severe: N=24.

FIG. 11: Inflammatory biomarkers as predictors of TBI outcome. Box and whisker plots showing the protein levels in pg/mL of caspasea-1 (A) and IL-10 (B) as a biomarker of outcome after TBI. Box and whiskers are shown for the 5th and 95th percentile. Dots correspond to data points outside the 5th and 95th percent confidence interval. ROC curves indicate the AUC (sensitivity vs 1-specificity) for (C) caspase-1: Favorable: N=47, Unfavorable: N=29 and (D) IL-10: Favorable: N=19, Unfavorable: N=11.

FIG. 12: Residual Analysis. Residual analysis of the multivariate linear regression models used to explain GCS (A) and GOS-E (B) were performed to determine the goodness of fit of each of the models.

FIG. 13: Inflammasome proteins are elevated after pTBI. Simple Plex Assay of blood serum from pTBI patients and age matched controls at 0, 24 and 48 hours. Inflammasome proteins that showed statistically significant increase after TBI were plotted. Box and whisker plots show mean, quartiles, and outliers for each inflammasome protein of interest with respective p values listed above. Dots correspond to data points outside the 5th and 95th percent confidence interval. Results showed A) ASC: N=Control: 22, pTBI at 0 h=31, 24 h=25 and 48 h=20; B) IL-10: N=Control=11, pTBI at 0 h=39, 24 h=27 and 48 h=24. ASC and IL-10 at 0 h were both significantly elevated in pTBI patients when compared to controls.

FIG. 14: ROC of inflammasome protein biomarkers. ROC and AUC were calculated for each inflammasome protein that were significantly different when comparing healthy uninjured controls and pTBI patients. A) ASC: N=Control=22, pTBI at 0 h=31, 24 h=25 and 48 h=20; B) IL-10=N=Control: 11, pTBI at 0 h=39, 24 h=27 and 48 h=24.

FIG. 15: Inflammatory biomarkers as predictors of TBI severity. A) Box and whisker plots showing the protein levels in pg/mL of ASC as a biomarker of injury severity at 0, 24 and 48 h. Box and whiskers are shown for the 5th and 95th percentile. Dots correspond to data points outside the 5th and 95th percent confidence interval. B) ROC curves indicate the AUC (sensitivity vs. 1-specificity) for ASC severity at 0, 24 and 48 h combined. ASC: N=Control=22, pTBI mild at 0 h=16, 24 h=8 and 48 h=7; pTBI moderate at 0 h=4, 24 h=5 and 48 h=4; pTBI severe at 0 h=11, 24 h=12 and 48 h=9.

FIG. 16: Inflammatory biomarkers as predictors of TBI outcome. (A) Box and whisker plots showing the protein levels in pg/mL of IL-18, 0 hr. after TBI at 12 months average. Box and whiskers are shown for the 5th and 95th percentile. Dots correspond to data points outside the 5th and 95th percent confidence interval. (B) ROC curves indicate the AUC (sensitivity vs 1-specificity) for IL-18, 0 h after TBI at 12 months average: Favorable: N=15, Unfavorable: N=5. (C) Box and whisker plots showing the protein levels in pg/mL of IL-18, 48 h after TBI at 12 months average. (D) ROC curves indicate the AUC (sensitivity vs 1-specificity) for IL-18, 48 h after TBI at 12 months average: Favorable: N=9, Unfavorable: N=5. (E) Box and whisker plots showing the protein levels in pg/mL of IL-1β, 0 h after TBI at 12 months average. (F) ROC curves indicate the AUC (sensitivity vs 1-specificity) for IL-1β, 0 h after TBI at 12 months average: Favorable: N=13, Unfavorable: N=5.

### DETAILED DESCRIPTION OF THE INVENTION

The present invention is based, in part, on the discovery that NLRP1 inflammasomes play an important role in inflammatory responses after SCI and TBI in humans. In particular, the present inventors have surprisingly found that nucleotide-binding leucine-rich repeat pyrin domain containing protein 1 (NLRP1), the adaptor protein apoptosis-associated speck-like protein containing a caspase recruitment domain (ASC), and caspase-1 are secreted into the cerebrospinal fluid (CSF) of human patients following SCI and TBI. Thus, these inflammasome proteins represent sensitive biomarkers of the severity of central nervous system injury in human patients. Accordingly, the present invention provides a method of assessing the severity of a central nervous system injury in a patient by measuring the level of at least one inflammasome protein in a biological sample obtained from the patient, wherein the measured level of said at least one inflammasome protein is indicative of the severity of the central nervous system injury in the patient.

As used herein, the term “inflammasome” refers to a multi-protein complex that activates caspase-1 activity, which in turn regulates IL-10, IL-18 and IL-33 processing and activation. See Arend et al. 2008; Li et al. 2008; and Martinon, et al. 2002, each of which is incorporated by reference in their entireties. An “inflammasome protein” is a protein component of inflammasome complexes and can include, but is not limited to, a nucleotide binding domain, leucine-rich repeat containing (NLR) family member (e.g., NLRP1), ASC, caspase-1, caspase-11 X-linked inhibitor of apoptosis protein (XTAP), and pannexin-1. NLRP1 is also known as NAcht leucine-rich-repeat protein 1 (NALP-1). Thus, the terms “NLRP1” and “NALP-1” are used interchangeably throughout the disclosure. In certain embodiments, the method comprises measuring an inflammasome protein selected from the group consisting of NLRP1 (NALP-1), ASC, caspase-1, or combinations thereof. In one embodiment, the p20 subunit of active caspase-1 is measured.

The terms “patient” or “subject” are used interchangeably herein, and is meant a mammalian subject to be treated, with human patients being preferred. In certain embodiments, the patient is a pediatric patient. Pediatric patients include newborns (birth to 1 month of age), infants (1 month to 2 years of age), children (2 to 12 years of age), and adolescents (12-21 years of age). In some cases, the methods of the invention find use in experimental animals, in veterinary application, and in the development of animal models for disease, including, but not limited to, rodents including mice, rats, and hamsters, and primates.

In certain embodiments, the present invention provides a method of evaluating a patient suspected of having a central nervous system (CNS) injury. In one embodiment, the method comprises providing a biological sample from a patient presenting with clinical symptoms consistent with a CNS injury; measuring the level of at least one inflammasome protein in the biological sample; determining the presence or absence of a protein signature associated with a CNS injury or a more severe CNS injury, wherein the protein signature comprises an elevated level of said at least one inflammasome protein; and selecting patients exhibiting the presence of the protein signature as having a CNS injury or a more severe CNS injury.

A patient may be suspected of having a CNS injury based on neurologic symptoms (motor, sensory, cognitive) and/or radiological evaluation (MRI, CT scan, X-ray) consistent with a CNS injury, e.g., after a physician's exam. In some embodiments, a patient suspected of having a CNS injury, particularly a spinal cord injury, may having a rating of A or Bon the American Spinal Cord Injury Association (ASIA) Impairment Scale. The ASIA Impairment Scale is a standard diagnostic tool that assesses a patient's motor and sensory function. The classification ratings and accompanying descriptions of the ASTA Impairment Scale are as follows:

| Classification/ | |
|---|---|
| Rating | Description |
| A | Complete: no motor or sensory function is preserved |
| | below the level of injury, including the sacral segments |
| | S4-S5 |
| B | Incomplete: sensory, but not motor, function is preserved |
| | below the neurologic level and some sensation in |
| | the sacral segments S4-S5 |
| C | Incomplete: motor function is preserved below the |
| | neurological level, however, more than half of key |
| | muscles below the neurologic level have a muscle |
| | grade less than 3 (i.e., not strong enough to move |
| | against gravity) |
| D | Incomplete: motor function is preserved below the |
| | neurologic level, and at least half of key muscles below |
| | the neurological level have a muscle grade of 3 or |
| | more (i.e., joints can be moved against gravity) |
| E | Normal: motor and sensory functions are normal |

Thus, a patient presenting with a classification rating of A or B on the ASIA Impairment Scale has no motor function below the level of the injury.

In other embodiments, a patient suspected of having a CNS injury may have a score of:S 12 (e.g., 3 to 12) on the Glasgow Coma Scale (GCS). In still other embodiments, the patient may have a GCS score of:S 8 (e.g., 3 to 8). The GCS is a neurological scale commonly used to assess the level of consciousness of patients after injury or trauma. The scale is composed of three tests (eye, verbal, and motor responses), each of which is assigned a value on a scale up to 6. The three values separately as well as their sum are considered. The lowest possible GCS score (the sum) is 3 (deep coma or death), while the highest is 15 (fully awake person). A GCS score <9 is indicative of severe brain injury whereas a GCS score ≥13 is indicative of minor brain injury. A GCS score between 9-12 is generally indicative of a moderate brain injury.

A patient suspected of having a CNS injury may have one or more signs and symptoms of CNS injury, such as temporary loss of consciousness, confusion, disorientation, memory or concentration problems, headache, dizziness, loss of balance, nausea or vomiting, sensory disruptions (e.g., blurred vision, ringing in the ears, bad taste in the mouth, loss of sensation in limbs), loss of motor function, sensitivity to light or sound, mood changes or mood swings, depression or anxiety, fatigue, drowsiness, and sleep disturbances.

In some embodiments, the level, concentration, or abundance of one or more inflammasome proteins is measured in a biological sample obtained from a patient (e.g., a patient suspected of having or suffering from a CNS injury). In particular embodiments, the levels, concentrations, or abundance of one or more inflammasome proteins is indicative of the severity of CNS injury in the patient. A CNS injury includes, but is not limited to, a traumatic brain injury, a stroke-related injury, a cerebral aneurism-related injury, a spinal cord injury (e.g., contusions, compressions, lacerations), concussion-related injury (including post-concussion syndrome), cerebral ischemia, injury resulting from neurodegenerative diseases (including Parkinson's disease, Dementia Pugilistica, Huntington's disease, Alzheimer's disease, Creutzfeldt-Jakob disease), seizure-related injuries, multiple sclerosis, amyotrophic lateral sclerosis, and other CNS traumas. In certain embodiments, the levels, concentrations, or abundance of one or more inflammasome proteins is indicative of the severity of traumatic brain injury or spinal cord injury in the patient.

As used herein, “biological sample” refers to any bodily fluid or tissue obtained from a patient or subject. A biological sample can include, but is not limited to, whole blood, red blood cells, plasma, serum, peripheral blood mononuclear cells (PBMCs), urine, saliva, tears, buccal swabs, CSF, CNS microdialysate, and nerve tissue. In one embodiment, the biological sample is CSF, saliva, serum, plasma, or urine. In certain embodiments, the biological sample is CSF.

In some embodiments, the measured level, concentration, or abundance of one or more inflammasome proteins in the biological sample is used to prepare an inflammasome protein profile, wherein the profile is indicative of the severity of a CNS injury in the patient or the patient's prognosis or recovery potential from a CNS injury. The inflammasome protein profile may comprise the level, abundance, or concentration of one or more inflammasome proteins measured in the patient's sample optionally in relation to a pre-determined value or range of reference values as described herein. In certain embodiments, the inflammasome proteins in the profile include NLRP1 (NALP-1), ASC, and/or caspase-1 (e.g., p20 subunit of caspase-1). In one particular embodiment, the inflammasome protein profile comprises the level, abundance, or concentration of each of NLRPI (NALP-1), ASC, and caspase-1 (e.g., p20 subunit of caspase-1).

In one aspect of the invention, the method of evaluating a patient suspected of having a CNS injury comprises determining the presence or absence of a protein signature associated with a CNS injury or a more severe CNS injury based on the measured level, abundance, or concentration of one or more inflammasome proteins in the patient sample or on the inflammasome protein profile prepared from the patient's sample. In certain embodiments, the protein signature comprises an elevated level of at least one inflammasome protein. The level of said at least one inflammasome protein in the protein signature may be enhanced relative to the level of the protein in a control sample or relative to a pre-determined reference value or range of reference values as further described herein. The protein signature may, in certain embodiments, comprise an elevated level for each of caspase-1 (e.g., p20 subunit of caspase-1), NLRP1, and ASC. Patients who exhibit the protein signature may be selected or identified as having a CNS injury or a more severe CNS injury.

The level or concentration of at least one inflammasome protein can be assessed at a single time point (e.g., after a potential CNS injury) and compared to a pre-determined reference value or range of reference values or can be assessed at multiple time points (e.g., two, three, four, five or more) after a potential CNS injury and compared to a pre-determined reference value or to previously assessed values. For instance, a biological sample for measuring levels or concentrations of inflammasome proteins can be obtained from a patient within one hour of a potential CNS injury to two weeks following a potential CNS injury. In some embodiments, the biological sample is obtained within one day, two days, three days, four days, five days, six days, seven days, ten days, or twelve days of a CNS injury or potential injury.

As used herein, “pre-determined reference value” refers to a pre-determined value of the level or concentration of an inflammasome protein ascertained from a known sample. For instance, the pre-determined reference value can reflect the level or concentration of an inflammasome protein in a sample obtained from a control subject (i.e., an uninjured, healthy subject). The control subject may, in some embodiments, be age-matched to the patients being evaluated. Thus, in particular embodiments, the measured level or concentration of at least one inflammasome protein is compared or determined relative to the level or concentration of said at least one inflammasome protein in a control sample (i.e. obtained from an uninjured subject).

In other embodiments, the pre-determined reference value or range of reference values can reflect the level or concentration of an inflammasome protein in a sample obtained from a patient with a known severity of CNS injury as assessed by clinical measures or post-mortem analysis. A pre-determined reference value can also be a known amount or concentration of an inflammasome protein. Such a known amount or concentration of an inflammasome protein may correlate with an average level or concentration of the inflammasome protein from a population of control subjects or a population of patients with known levels of injury. In another embodiment, the pre-determined reference value can be a range of values, which, for instance, can represent a mean plus or minus a standard deviation or confidence interval. A range of reference values can also refer to individual reference values for a particular inflammasome protein across various levels of CNS injury severity. In certain embodiments, an increase in the level of one or more inflammasome proteins (e.g., NLRP1 (NALP-1), ASC, or caspase-1) relative to a pre-determined reference value or range of reference values is indicative of a more severe central nervous system injury.

In some embodiments, the method of assessing the severity of a CNS injury further comprises measuring the level or concentration of one or more proteins described in U.S. Patent Publication No. 2011/0177974, which is hereby incorporated by reference in its entirety, in addition to measuring the level or concentration of one or more inflammasome proteins. For instance, in certain embodiments, the method further comprises measuring the level or concentration of one or more proteins selected from ubiquitin C-terminal hydrolase LI; vesicular membrane protein p-24; synuclein; micro tubule-associated protein; synaptophysin; Vimentin; Synaptotagmin; Synaptojanin-2; Synapsin2; CRMPI, 2; Amphiphysin-1; PSD95; PSD-93; Calmodulin dependent protein kinase TT (CAMPK)-alpha, beta, gamma; Myelin basic protein (MBP); Myelin proteolipid protein (PLP); Myelin Oligodendrocyte specific protein (MOSP); Myelin Oligodendrocyte glycoprotein (MOG); myelin associated protein (MAG); NF-H; NF-L; NF-M; Bill-tubulin-1 or combinations thereof in the biological sample obtained from the patient in addition to measuring the level or concentration of one or more inflammasome proteins. Thus, the protein signature may comprise an elevated level of one or more of these proteins in addition to the elevated level of one or more inflammasome proteins. In other embodiments, the method further comprises measuring the level or concentration of one or more proteins selected from S-10013, neuron-specific enolase, neurofilament light chain, glial fibrillary acidic protein (GFAP) or combinations thereof in the biological sample obtained from the patient in addition to measuring the level or concentration of one or more inflammasome proteins. In one embodiment, the protein signature associated with a CNS injury, or a more severe CNS injury, comprises an elevated level of one or more proteins selected from S-1000, neuron-specific enolase, neurofilament light chain, glial fibrillary acidic protein (GF AP) in addition to an elevated level of one or more inflammasome proteins (e.g., NLRP1 (NALP-1), ASC, or caspase-1).

In other embodiments of the invention, the methods of assessing the severity of a CNS injury in a patient or evaluating a patient suspected of having a CNS injury further comprise administering a neuroprotective treatment to the patient based on the measured level of said at least one inflammasome protein or when a protein signature associated with a CNS injury, or a more severe CNS injury, is identified. Such neuroprotective treatments include drugs that reduce excitotoxicity, oxidative stress, and inflammation. Thus, suitable neuroprotective treatments include, but are not limited to, methylprednisolone, 17a-estradiol, 170-estradiol, ginsenoside, progesterone, simvastatin, deprenyl, minocycline, resveratrol, and other glutamate receptor antagonists (e.g., NMDA receptor antagonists) and antioxidants.

The success of, or response to, treatment can also be monitored by measuring the levels of at least one inflammasome protein. Accordingly, in some embodiments, the methods of evaluating a patient further comprise measuring the level of at least one inflammasome protein in a biological sample obtained from the patient following neuroprotective treatment, preparing a treatment protein signature associated with a positive response to the neuroprotective treatment, wherein the treatment protein signature comprises a reduced level of at least one inflammasome protein, and identifying patients exhibiting the presence of the treatment protein signature as responding positively to the neuroprotective treatment. A reduction in the level, abundance, or concentration of one or more inflammasome proteins (e.g., NLRP1, ASC, and caspase-1) is indicative of the efficacy of the neuroprotective treatment in the patient. The one or more inflammasome proteins measured in the sample obtained following treatment may be the same as or different than the inflammasome proteins measured in the sample obtained prior to treatment. The inflammasome protein levels may also be used to adjust dosage or frequency of a neuroprotective treatment.

The present invention also provides a method of determining a prognosis for a patient with a central nervous system injury. In one embodiment, the method comprises providing a biological sample obtained from the patient within a week of injury and measuring the level of at least one inflammasome protein in the biological sample to prepare an inflammasome protein profile as described above, wherein the inflammasome protein profile is indicative of the prognosis of the patient. In certain preferred embodiments, the biological sample is obtained from the patient within one week, within five days, or within three days of injury. In some embodiments, an increase in the level of one or more inflammasome proteins (e.g., NLRP1, ASC, caspase-1, or combinations thereof) relative to a pre-determined reference value or range of reference values is indicative of a poorer prognosis. For instance, an increase of about 20% to about 300% in the level of one or more inflammasome proteins relative to a pre-determined reference value or range of reference values is indicative of a poorer prognosis. In one embodiment, increased levels of caspase-1, particularly the p20 subunit of active caspase-1, relative to a pre-determined reference value or range of reference values acutely after injury (i.e., within a week of injury) is indicative of a poorer prognosis.

In particular embodiments, an elevated level of at least one inflammasome protein relative to a pre-determined reference value or range of reference values is predictive of the patient's recovery potential or long-term outcome as assessed by the Glasgow Outcome Scale (GOS). The GOS is a scale that allows for the objective assessment of a patient's recovery following brain injury. The scale is comprised of scores ranging from 1 to 5 with the following descriptions:

| Score/Category | Description |
|---|---|
| 1-Death | Severe injury or death without recovery of |
| | consciousness |
| 2-Persistent | Severe damage with prolonged state of |
| Vegetative State | unresponsiveness and a lack of higher mental |
| | functions |
| 3-Severe | Severe injury with permanent need for help with |
| Disability | daily living |
| 4-Moderate | No need for assistance in everyday life, employment |
| Disability | is possible but may require special equipment |
| 5-Low Disability | Light damage with minor neurological and |
| | psychological deficits. |

In one embodiment, an elevated level of at least one inflammasome protein relative to a pre-determined reference value or range of reference values is predictive of the patient having a GOS score of 1 to 3 upon follow-up assessment (i.e., the patient having an unfavorable outcome, such as death or severe disability). In another embodiment, a reduced level of at least one inflammasome protein relative to a pre-determined reference value or range of reference values is predictive of the patient having a GOS score of 4 or 5 upon follow-up assessment (i.e., the patient having a favorable outcome, such as moderate to low disability). The inventors have found that the CSF levels of one or more inflammasome proteins within three days following a CNS injury are useful for predicting the long-term outcome or recovery potential of the patient. Elevated inflammasome proteins levels correlate with unfavorable outcomes for the patient, whereas reduced or low inflammasome protein levels correlate with favorable outcomes for the patient (Example 3).

The inflammasome proteins of the invention and other marker proteins can be measured in a biological sample by various methods known to those skilled in the art. For instance, proteins can be measured by methods including, but not limited to, liquid chromatography, gas chromatography, mass spectrometry, radioimmunoassays, immunofluorescent assays, FRET-based assays, immunoblot, ELTSAs, or liquid chromatography followed by mass spectrometry (e.g., MALDI MS). One of skill in the art can ascertain other suitable methods for measuring and quantitating any particular biomarker protein of the invention.

In accordance with the subject invention, immune proteins or inflammasomes, or inflammasome component proteins which are present at certain levels in a tissue sample of the patient, can establish whether a patient having a CNS injury is diagnosed as having traumatic brain injury (TBI). These levels are cut-off values for the determination of TBI, the diagnosis of which can be predictive of prognosis of the patient, and can be used to initiate protective or therapeutic intervention, such as administration of one or more drug (e.g., small molecule) or biologic (e.g., large molecule peptide, protein, or antibody) useful to protect the patient from further brain injury or reduce the symptoms of TBI.

For adult patients with brain injury, biomarkers and cut-off values (in serum) which are useful in accordance with the invention to determine traumatic brain injury (TBI), include: Caspase-1: >0.8150 pg/ml; ASC: >284 pg/ml; IL-18: >156 pg/ml; TNF-α: >2.202 pg/ml; IL-6: >6.443 pg/ml; IL-4: >0.03868 pg/ml; IL-10: >0.6527 pg/ml; IL-8: >29.18 pg/ml, and IL-2: <0.5145 pg/ml. A value within twenty percent (±20%) of any of the given value is considered equivalent to the given value.

In pediatric patients with brain injury, biomarkers and cut-off values (in serum) which are useful in accordance with the invention to determine traumatic brain injury (TBI), include: Caspase-1: >2.51 pg/ml; ASC: >469.5 pg/ml; IL-18: <256 pg/ml; and IL-10: <0.57 pg/ml. A value within twenty percent (±20%) of any of the given value is considered equivalent to the given value.

In some embodiments, neuroprotective treatments are neutralizing antibodies against an inflammasome protein or binding fragments thereof, such as those described in U.S. Patent Publication No. 2009/0104200, which is hereby incorporated by reference in its entirety. For instance, in one embodiment, the neuroprotective treatment is an anti-ASC antibody or fragment thereof. Anti-ASC antibodies include antibodies that specifically bind to amino acid residues 178-193 of rat ASC (accession number BAC43754), e.g., amino acid sequence ALRQTQPYLVTDLEQS (SEQ ID NO:1), or antibodies that specifically bind to the amino acid sequence RESQSYLVEDLERS (SEQ ID NO:2) of human ASC. In another embodiment, the neuroprotective treatment is an anti-NLRP1 antibody or fragment thereof. Suitable neutralizing anti-NLRP1 antibodies or fragments thereof include antibodies that specifically bind to the amino acid sequence CEYYTEIREREREKSEKGR (SEQ ID NO:3) of human NLRP1 or the amino acid sequence MEESQSKEESNTEG (SEQ ID NO: 4) of rat NLRP1. The neutralizing antibodies or antibody fragments may be polyclonal antibodies, monoclonal antibodies, chimeric antibodies, humanized antibodies, single-chain variable fragments (scFvs) and the like. Aptamers that specifically bind to an inflammasome protein or epitope thereof (e.g., SEQ ID NOs: 1-4) may also be suitable neuroprotective treatments. Neuroprotective treatments also encompass therapeutic regimens or rehabilitative procedures, such as hypothermia treatment.

Neutralizing antibodies against an inflammasome protein or binding fragments thereof, can include any one of, or two or more in combination, of the following: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; or SEQ ID NO:41; in the Sequence Listing provided as part of this description and incorporated herein by reference. Pharmaceutical compositions comprising one or more of the listed antibodies of SEQ ID NOs 1-41, one or more other antibody known or discovered to bind the biomarker molecule, or one or more small molecule drug known or discovered to inactivate the biomarker molecule, can be used in a pharmaceutically effective amount as an active component of the pharmaceutical composition and formulated for administration to a human or animal patient with one or more pharmaceutically acceptable solvent, excipient, or carrier.

The present invention also includes kits for preparing an inflammasome protein profile associated with CNS injury, such as spinal cord injury or traumatic brain injury. The kits may include a reagent for measuring at least one inflammasome protein and instructions for measuring said at least one inflammasome protein for assessing the severity of a central nervous system injury in a patient. As used herein, a “reagent” refers to the components necessary for detecting or quantitating one or more proteins by any one of the methods described herein. For instance, in some embodiments, kits for measuring one or more inflammasome proteins can include reagents for performing liquid or gas chromatography, mass spectrometry, immunoassays, immunoblots, or electrophoresis to detect one or more inflammasome proteins as described herein. In some embodiments, the kit includes reagents for measuring one or more inflammasome proteins selected from NLRP1, ASC, caspase-1, or combinations thereof.

In one embodiment, the kit comprises a labeled-binding partner that specifically binds to one or more inflammasome proteins, wherein said one or more inflammasome proteins are selected from the group consisting of NLRP1, ASC, caspase-1, and combinations thereof. Suitable binding partners for specifically binding to inflammasome proteins include, but are not limited to, antibodies and fragments thereof, aptamers, peptides, and the like. In certain embodiments, the binding partners for detecting NLPR I are antibodies or fragments thereof, aptamers, or peptides that specifically bind to the amino acid sequence of SEQ ID NO: 3 or SEQ ID NO: 4 of human NLRP1 and rat NLRP1, respectively. In other embodiments, the binding partners for detecting ASC arc antibodies or fragments thereof, aptamers, or peptides that specifically bind to the amino acid sequence of SEQ ID NO: 1 or SEQ ID NO: 2 of rat ASC and human ASC, respectively. Labels that can be conjugated to the binding partner include metal nanoparticles (e.g., gold, silver, copper, platinum, cadmium, and composite nanoparticles), fluorescent labels (e.g., fluorescein, Texas-Red, green fluorescent protein, yellow fluorescent protein, cyan fluorescent protein, Alexa dye molecules, etc.), and enzyme labels (e.g., alkaline phosphatase, horseradish peroxidase, beta-galactosidase, beta-lactamase, galactose oxidase, lactoperoxidase, luciferase, myeloperoxidase, and amylase).

In some embodiments, the kit can include reagents for measuring one or more inflammasome proteins in CSF samples. In other embodiments, the kits can include reagents for measuring one or more inflammasome proteins in other patient samples including nerve tissue, CNS microdialysate, blood, saliva, serum, plasma, or urine. In still other embodiments, the kits further comprise a set of reference values to which the measured level of one or more inflammasome proteins can be compared.

Biomarkers are specific proteins that are used as indicators of the status of different physiological processes in an individual. Biomarkers are often used to determine the stage or severity of an underlying disease or injury. TBI presents as a multi-factorial series of events that affect a variety of cells within the central nervous system (CNS) including neurons, microglia, astrocytes, oligodendrocytes and endothelial cells. Thus, a variety of biomarkers need to be identified in order to have a better understanding of the different molecular events that affect different cell types after TBI in the clinical setting. To this point, two biomarkers have been approved by the FDA for the monitoring of TBI patients. These biomarkers are Ubiquitin carboxy-terminal hydrolase (UCH-L1) and glial fibrillary acidic protein (GFAP). UCH-L1 is expressed in neurons, and it is highly upregulated after TBI, whereas GFAP is similarly expressed in astrocytes. Thus, these two FDA approved biomarkers for TBI offer clinicians an insight as to the degree of neuronal degradation and astroglial activation after brain injury. However, to date, no approved fluid biomarker or series of biomarkers is available to determine the inflammatory response in the acute setting after TBI.

Previous studies have shown that inflammasome proteins are potentially effective indicators of TBI severity and pathological outcomes in TBI. Levels of ASC and caspase-1 are elevated in the blood of TBI patients with increased ASC levels correlating with more severe injury and worse outcomes. Additionally, ASC and IL-18 were elevated in the cerebral spinal fluid (CSF) of patients after TBI, and caspase-1 correlated with increased intracranial pressure and poor outcomes. Moreover, several inflammatory cytokines have been described in the literature as potential biomarkers of TBI. For instance, tumor necrosis factor-α (TNF-α), IL-8 and IL-10 have been described to be elevated in the CSF and serum of patients with TBI, and protein levels of IL-6 in plasma have been shown to correlate with brain injury severity.

Despite ample evidence for increased expression of a variety of inflammatory proteins in the CSF and blood of patients with TBI when compared to healthy uninjured controls, few studies have aimed to determine the biomarker characteristics of these inflammatory proteins including the receiver operating characteristic (ROC) curve as well as the determination of cut-off points to identify the respective sensitivity and specificity of the different inflammatory biomarkers. In addition, previous studies have not compared the area under the curve (AUC) between different inflammatory biomarkers with the goal of identifying which inflammatory biomarkers are more suitable surrogates of the inflammatory response taking place acutely after TBI. Thus, in this study we measure the protein levels of inflammasome signaling proteins and inflammatory cytokines associated with TBI to then determine the biomarker characteristics of these proteins as well as the contribution of these inflammatory proteins to long term outcomes as determined by the Glasgow-Outcome Scale-Extended (GOS-E) and to injury severity as determined by the Glasgow-Comma Scale (GCS). Importantly, here we follow a systematic approach to determine the suitability of each biomarker as a surrogate of inflammation following TBI and compare the AUC between each biomarker to identify which biomarkers among all the promising inflammatory biomarkers analyzed in this study have the potential to be more reliable biomarkers that can be used in the clinical setting.

This invention is further illustrated by the following additional examples that should not be construed as limiting. Those of skill in the art should, in light of the present disclosure, appreciate that many changes can be made to the specific embodiments which arc disclosed and still obtain a like or similar result without departing from the spirit and scope of the invention.

### EXAMPLES

### Example 1. Inflammasome Proteins are Secreted into Cerebrospinal Fluid after Spinal Cord Injury

To determine whether NLRP1 inflammasome proteins were present in cerebrospinal fluid (CSF) following spinal cord injury (SCI), CSF samples from seven patients with SCI or control patients were analyzed for levels of nucleotide-binding leucine-rich repeat pyrin domain containing protein 1 (NLRP1; also known as NAcht leucine-rich-repeat protein 1 (NALP-1)), apoptosis-associated speck-like protein containing a caspase recruitment domain (ASC), and caspase-1. The American Spinal Cord Injury Association (ASTA) scale of the SCT patients at admission to the emergency department ranged from AIS A to B. Information regarding the diagnosis, procedures and outcomes of the patients is shown in Table 1. None of the patients had any complications. CSF from uninjured individuals was obtained as a control from three males and two females ranging from 67 to 91 years old.

TABLE 1
Spinal Cord Injury Subjects

| | | | | Mechanism | Spinal | AIS | | | | Other | Exam at |
|---|---|---|---|---|---|---|---|---|---|---|---|
| Patient | Age | Gender | Race | of Injury | Injury | Grade | Level | Surgery | Hypothermia | injuries | Rehab D/C |
| 1 | 43 | M | Black | Auto vs. | C7/T1 | A | C7 | C4-T2 | Yes | None | C7 |
| | | | | Pedestrian | bilateral | | | Laminectomy | | | ASIA A |
| | | | | | jumped | | | w/instrumented | | | |
| | | | | | facets | | | fusion | | | |
| 2 | 38 | M | Hispanic | Fall from | T3 & T4 | B | T3 | T1-6 | No | None | T3 |
| | | | | height | Burst | | | Laminectomy | | | ASIA D |
| | | | | | fractures | | | w/instrumented | | | |
| | | | | | | | | fusion | | | |
| 3 | 21 | M | White | Driving | C5/6 | A | C5 | C5/6 Anterior | Yes | None | C5 |
| | | | | accident | Fracture | | | disectomy & | | | ASIA C |
| | | | | | dislocation | | | fusion | | | |
| 4 | 19 | M | Black | Motor | C4/5 | B | C4 | C4/5 | No | None | C4 |
| | | | | vehicle | Bilateral | | | Anterior | | | ASIA D |
| | | | | accident | jumped | | | disectomy & | | | |
| | | | | | facets | | | fusion | | | |
| 5 | 22 | M | White | Rugby | C5/6 | A | C5 | C5/6 Anterior | Yes | None | C6 |
| | | | | accident | Bilateral | | | disectomy & | | | ASIA A |
| | | | | | jumped | | | fusion | | | |
| | | | | | facets | | | | | | |
| 6 | 48 | M | Hispanic | Motorcycle vs. | T1/2 | A | T3 | C5-T2 | No | Degloving | T3 |
| | | | | Pedestrian | Fracture | | | Laminectomy | | injury and | ASIA B |
| | | | | | dislocation | | | w/instrumented | | fractures | |
| | | | | | | | | fusion | | of face | |

For detection of inflammasome proteins, CSF samples were prepared with Laemali buffer. Immunoblot analysis was carried out with the Criterion system (Bio-Rad) as described previously (de Rivero Vaccari et al., 2008) using antibodies (1:1000 dilution) to NLRP1 (Bethyl Laboratories), Caspase-1 (Imgenex) and ASC (Santa Cruz). Proteins were resolved in 14-20% TGX Criterion precasted gels (Bio-Rad), transferred to polyvinylidene difluoride (PVDF) transfer membranes (Tropifluor-Applied Biosystems) and placed in blocking buffer (PBS, 0.1% Tween-20, 0.4% 1-Block (Applied Biosystems) and then incubated for one hour with primary antibodies. Membranes were then incubated for one hour with anti-mouse, anti-rat, or anti-rabbit horseradish peroxidase (HRP)-linked antibodies. Signal visualization was performed by enhanced chemiluminescence.

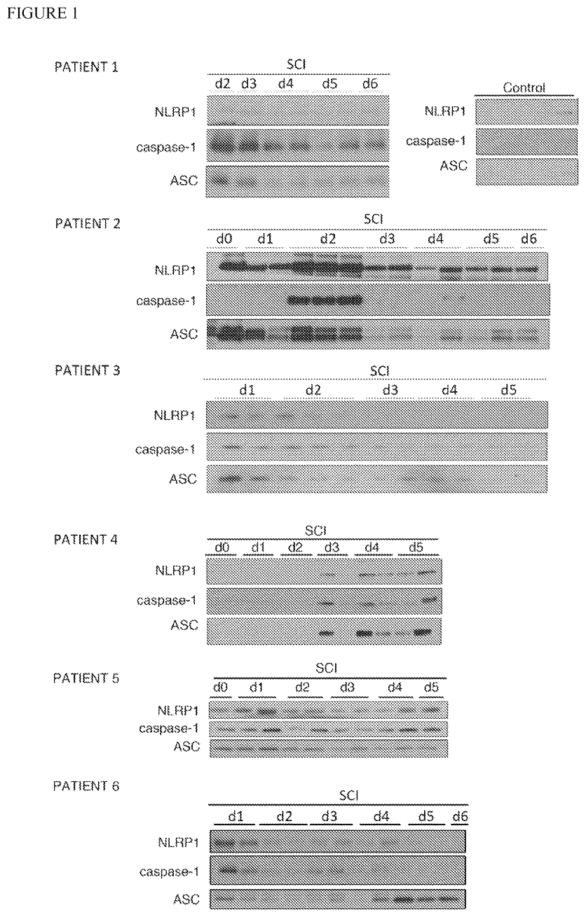

Immunoblot analysis of control CSF samples (n=5) revealed very low levels of NLRP1 inflammasome proteins (FIG. 1). In contrast, immunoblot analysis of samples from 6 different SCI patients showed an increase in the levels of NLRP1, caspase-1, and ASC in the CSF when compared to CSF from control subjects. It should be noted that patients 2, 3 and 4 (FIG. 1) did not show increased levels of caspase-1 acutely (day 0 through day 2) after SCI. Interestingly, these patients demonstrated stark motor improvement at 2 days after SCI. Patients 1, 5, and 6 showed increased levels of caspase-1, ASC and NLRP1 inflammasome proteins acutely after SCI and these individuals had a poor prognosis and did not show motor improvements. Thus, it appears that individuals that present with low levels of caspase-1 in CSF acutely after SCI may have a better prognosis than those individuals who show increased levels of this biomarker.

The results from these experiments show that protein levels of NLRP1, ASC, and caspase-1 in CSF are increased following injury to the central nervous system and suggest that levels of these inflammasome proteins can serve as biomarkers of the severity of neuronal damage following injury thereby directing treatment and rehabilitation efforts, monitoring response to treatment, and aiding in the determination of prognosis of recovery in injured patients.

### Example 2. Immunohistochemical Expression of NLRP1 Inflammasome Proteins in Spinal Cords After Injury

Spinal cord sections were obtained from nine decedents (8 males and 1 female with ages ranging from 20 to 77 years) who had injury to the spinal cord due to vertebral fractures. The spinal cord injury was assessed microscopically, using bright field optics, by examining one H&E or H&E/DAB-stained section from the lesion center of each case or from cervical, thoracic and lumbar sections from control cases. The spinal cord injuries were classified based on their histological appearance as “contusion/cyst,” massive compression, or laceration (Fleming et al., 2006). Contusional injuries were characterized by an intact pia and relative preservation of the anatomical relations of various elements of the spinal cord, and variable degrees of injury ranging from involvement of the entire cross-sectional area to large usually asymmetric areas of tissue damage. Massive compression injuries were characterized by disruption of the pia and severe distortion and disruption of spinal cord parenchyma. Laceration injuries which by definition were perforating or penetrating injuries caused by weapons or projectiles, were associated with breaching of the pia and linear tearing of the cord tissue.

All tissue samples had been removed within 24 h of death and fixed in neutral buffered formalin. Blocks from the spinal cords were dehydrated, embedded in paraffin wax, cut into 6 μm thick sections and placed on positively charged glass slides. One set of sections was stained with hematoxylin-eosin (H&E) and the remaining sets were used for immunohistochemistry. Paraffin-embedded sections were stained with anti-NLRP1 (Bethyl Laboratories as described in de Rivero Vaccari et al. 2008), anti-caspase-1 (Upstate), and anti-ASC (Chemicon) using diaminobenzidine (DAB) as the chromophore and hematoxylin. Negative controls included sections in which the primary antibody was omitted, and sections incubated with isotype-matched antibodies (1:100-1:10,000 IgG). These positive and negative controls were processed with every batch of immunohistochemical slides.

In all cases, tissue samples from the center of injury and at various distances above and below the injury were obtained. The data from tissue from the center of the lesion were used to compare the inflammatory responses between cases whereas those from the remote, uninjured segments of the spinal cord served as within-case controls. Between-case comparison of the remote samples was not possible because, for different cases, the distance of these samples from the lesion center was variable.

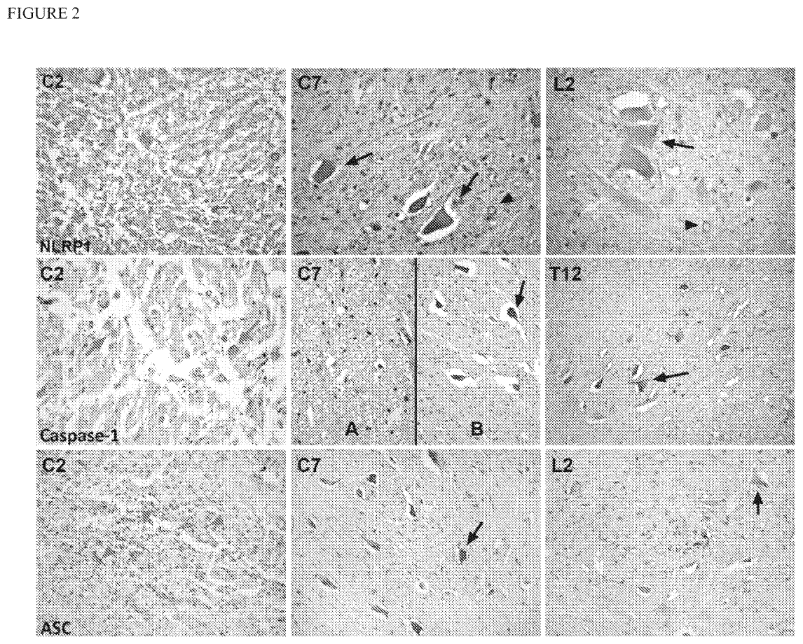

Immunohistochemical analysis combined with light microscopy indicated that NLRP1 is expressed in neurons of the ventral horn (black arrows), myelinated axons (arrow heads) and oligodendrocytes (yellow arrows) in injured spinal cords (FIG. 2). Moreover, NLRP1 immunoreactivity in areas of the penumbra (C7) was higher than in areas distant to the epicenter (12).

DAB immunoreactivity for caspase-1 was detected in swollen axons (spheroids, blue arrows) (FIG. 2), and arterioles (not shown). At areas of the penumbra, caspase-1 staining is present in motor neurons (black arrows) of the ventral horn, and in the white matter in oligodendrocytes (yellow arrows). Caspase-1 immunoreactivity in oligodendrocytes (yellow arrows) was the same at all levels of the spinal cords examined, regardless of proximity to the epicenter. At areas distant to the epicenter (T12), caspase-1 was also present in motor neurons (black arrows) but with decreased immunoreactivity than the penumbra (C7). This finding indicates that caspase-1 immunoreactivity in neurons decreases as the distance to the epicenter increases, similarly to NLRP1.

At areas of the penumbra (C7) and distant to the epicenter (12), neurons in the ventral horn (black arrows) and white matter oligodendrocytes (yellow arrow) showed ASC immunoreactivity. In addition, ASC was also present in macrophages/microglia at the epicenter (blue arrow heads). Moreover, ASC immunoreactivity was also detected in the substantia gelatinosa (dorsal horn) at C7 and L2 (not shown).

Neuroinflammation has been considered to playa critical role in the pathogenesis of SCI and TBI, but the role of the innate immune response has not been examined directly. The innate immune system senses microbial and viral pathogen-associated molecular patterns and danger signals released from damaged or stressed cells to trigger conserved intracellular signaling pathways that drive proinflammatory responses that are critical for productive innate and adaptive immunity. Excessive inflammatory responses become deleterious leading to tissue destruction. The results of this experiment provide evidence demonstrating that the NLRP1 inflammasome signaling system is activated in the innate immune response in damaged human spinal cord and brain tissue after trauma. These findings support the idea that activation of the NLRP1 inflammasome signaling system is an early event in spinal cord and brain pathology and that these proteins may serve as biomarkers for SCI and TBI in humans.

### Example 3. Inflammasome Proteins in Cerebrospinal Fluid of Brain-Injured Patients are Biomarkers of Functional Outcome

To determine whether inflammasome proteins may serve as biomarkers for other types of central nervous system injury, a total of 45 CSF samples were collected from 23 traumatic brain injury (TBI) patients on the day of injury and up to three days after the injury and analyzed by immunoblot for levels of NALP-1 (also known as NLRP1), ASC, and caspase-1.

Each of the patients presented with the following inclusion criteria: severe or moderate head trauma (Glasgow Coma Scale (GCS) score ≤12), age 1 month to 65 years, and ventriculostomy. Twenty-two of the patients suffered severe brain trauma (GCS score ≤8) and 1 suffered moderate brain trauma (moderate TBI GCS score range 9-12). Nine patients (5 men and 4 women) with a mean age of 66.3 years (range 29-91 years) served as controls. Control patients required a ventriculostomy for nontraumatic pathology. Patients with acute meningitis, cerebral vasculitis, or other recent CNS infection were excluded. Information regarding patient demographics, intracranial pathology, GCS score at presentation, and Glasgow Outcome Scale (GOS) score at 5 months post-injury is shown in Table 2.

TABLE 2
Summary of demographic data in patients with TBI*

| | Age | | | | | |
|---|---|---|---|---|---|---|
| | (yrs), | | Mechanism | GCS | GOS | |
| Case No. | Sex | Race | of Injury | Score† | Score‡ | Intracranial Pathology |
| 1 | 26, M | White | MVA | 3 | 4 | bilat temporal cortical contusions, SAH, SDH |
| 2 | 22, M | Hispanic | motorcycle | 3 | 4 | SDH, diffuse SAH |
| | | | accident | | | |
| 3 | 19, F | Hispanic | MVA | 7 | 3 | fronto- & temporoparietal & basal ganglia |
| | | | | | | hemorrhagic contusions, frontoparietal SAH |
| 4 | 30, F | Hispanic | MVA | 8 | 4 | scattered SAH, diffuse cerebral edema |
| 5 | 21, M | Hispanic | motorcycle | 5 | 3 | frontal SDH & SAH |
| | | | accident | | | |
| 6 | 17, F | Hispanic | MVA | 4 | 3 | diffuse extraaxial & intraparenchymal hemorrhage |
| 7 | 26, M | Hispanic | MVA | 8 | 3 | diffuse SAH, IVH, & parenchymal hemorrhagic |
| | | | | | | contusions |
| 8 | 36, M | White | ATV accident | 3 | 5 | SDH, SAH, & diffuse cerebral edema |
| 9 | 16, M | Hispanic | MVA | 7 | 5 | frontotemporal SDH, frontal lobe contusion, cerebral |
| | | | | | | edema |
| 10 | 22, M | Hispanic | MVA | 3 | 3 | SDH |
| 11 | 36, M | Black | Gunshot | 3 | 1 | SDH, bullet fragments in frontal lobe |
| | | | wound | | | |
| 12 | 54, M | Hispanic | Fall | 3 | 3 | frontal hemorrhagic contusions, parietooccipital |
| | | | | | | hemorrhagic contusion, SDH, brain edema |
| 13 | 62, M | Black | MVA | 8 | 3 | diffuse frontoparietal hemorrhage, frontal & parietal |
| | | | | | | lobe hemorrhagic contusions |
| 14 | 49, F | White | Assault | 8 | 3 | temporal & frontal lobe hemorrhagic contusions, |
| | | | | | | frontotemporal SAH, frontal dural hematoma |
| 15 | 20, M | Hispanic | Motorcycle | 6 | 4 | bone fragments in frontoparietal brain parenchyma, |
| | | | accident | | | scattered frontoparietal SAH, hemorrhagic contusions |
| 16 | 28, M | Hispanic | Motorcycle | 4 | 3 | SDH |
| | | | accident | | | |
| 17 | 21, M | Black | MVA | 5 | 4 | mild cerebral edema |
| 18 | 45, M | Hispanic | Gunshot | 8 | 3 | bullet fragments in occipital lobe, occipital SDH, |
| | | | wound | | | minimal parietal & occipital pneumocephalus |
| 19 | 21, M | White | MVA | 3 | 3 | diffuse axonal injury, scattered SAH, parietal SDH, |
| | | | | | | mild hydrocephalus |
| 20 | 17, M | Hispanic | Sports injury | 4 | 4 | SDH, SAH, diffuse cerebral edema |
| 21 | 19, F | Hispanic | MVA | 11 | 5 | bone fragments in frontal lobe parenchyma, frontal |
| | | | | | | lobe contusion, edema, pneumocephalus |
| 22 | 65, M | White | MVA | 7 | 1 | parietal SDH, SAH, parietal hemorrhagic contusion, |
| | | | | | | uncal herniation |
| 23 | 18, M | White | MVA | 3 | 1 | diffuse axonal injury, scattered hemorrhagic |
| | | | | | | contusions in frontal, temporal, parietal lobes & |
| | | | | | | corpus callosum, IVH |

*IVH = intraventricular hemorrhage; SAH = subarachnoid hemorrhage; SDH = subdural hematoma. MVA = motor vehicle accident; ATV = all terrain vehicle
†Obtained on admission.
‡Assessed at 5 months postinjury.

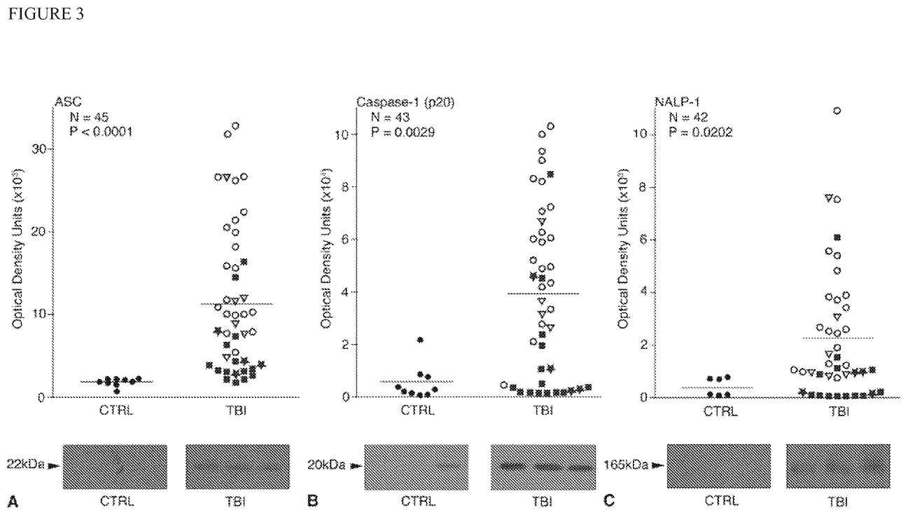

Cerebrospinal fluid samples were collected within 12 hours of injury and up to 72 hours after injury. Samples were centrifuged at 2000 g for 10 minutes at 4° C. to pellet cellular bodies and debris. Supernatants were resolved by gel electrophoresis and immunoblotted as previously described (de Rivero Vaccari et al., 2008). Quantification of band density was performed with UNSCAN-IT gel digitizing software (Silk Scientific). Due to the low volume of sample available, NALP-1 was analyzed in 6 of the 9 controls, caspase-1 was analyzed in 43 of the 45 TBI samples, and NALP-1 was analyzed in 42 of the 45 TBI samples. Immunoblot analysis shows that the inflammasome proteins ASC, caspase-1 (p20), and NALP-1 are present in the CSF of patients with TBI and nontrauma controls. Quantitative data from a densitometric analysis are shown in FIG. 3. Expression of the 22-kD isoform of ASC (FIG. 3A), the p20 subunit of cleaved caspase-1 (FIG. 3B), and NALP-1 (FIG. 3C) is significantly elevated in the CSF of TBI patients compared with nontrauma controls (p<0.0001, p=0.0029, and p=0.0202, respectively).

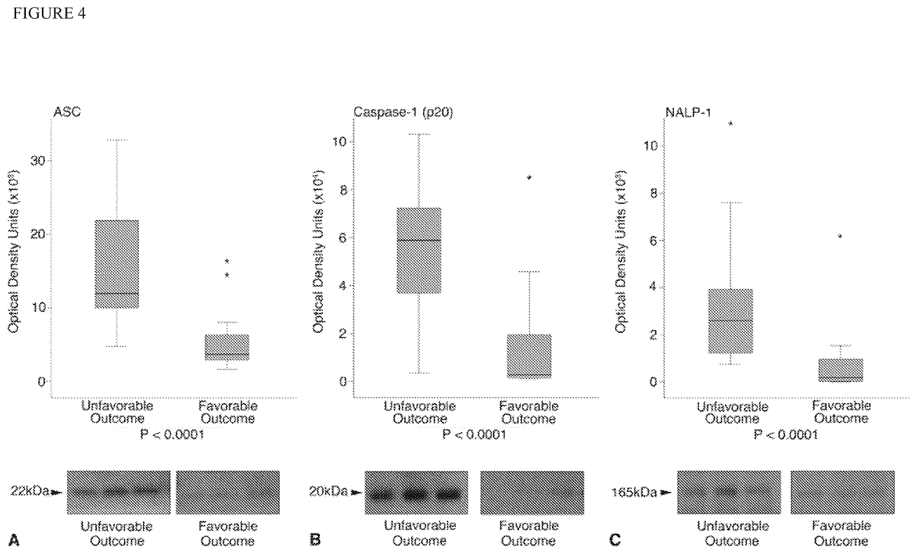

To determine if the levels of inflammasome components correlate with outcome, we grouped study participants by outcome category (GOS Scores 1 and 3, unfavorable outcome; GOS Scores 4 and 5, favorable outcome). At 5 months postinjury, 3 patients had a GOS score of 1 (death), 11 patients had a GOS score of 3 (severe disability), 6 patients had a GOS score of 4 (moderate disability), and 3 patients had a GOS score of 5 (good recovery). Within the sample of patients with TBI, no patient remained with a GOS score of 2 (persistent vegetative state). We detected significantly higher levels of ASC (FIG. 4A), caspase-1 (p20) (FIG. 4B), and NALP-1 (FIG. 4C) in the CSF of TBI patients with unfavorable outcomes, including death and severe disability with complete dependence on others for activities of daily living (p <0.0001).

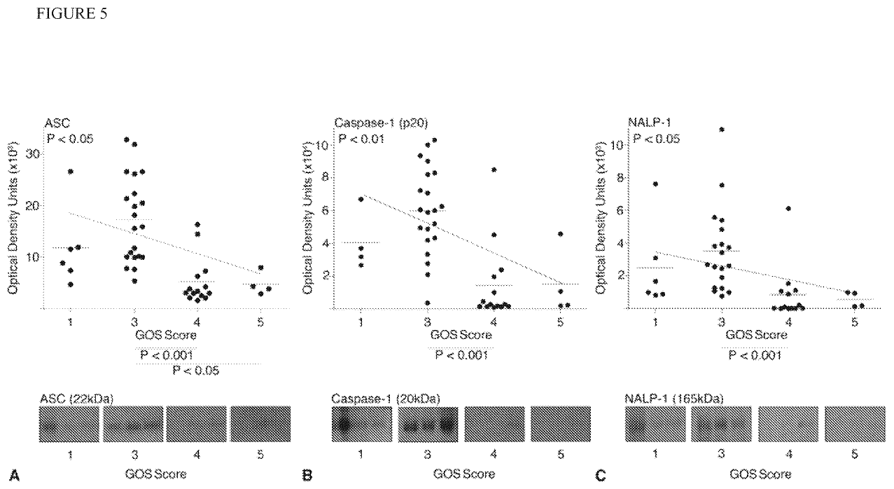

To further understand the relationship between outcome and inflammasome proteins, we constructed modified scatter plots of expression levels of ASC, caspase-1 (p20), and NALP-1 and GOS (FIG. 5). A calculated linear regression line is shown for each plot. Linear regression analysis shows that expression of ASC (FIG. 5A; p<0.05), caspase-1 (p20) (FIG. 5B; p<0.01), and NALP-1 (FIG. 5C; p<0.05) correlate significantly with outcome at 5 months. Post hoc, the Dunn multiple comparison tests following a Kruskal-Wallis test showed that the levels of ASC are significantly higher in patients with severe disability (GOS Score 3) compared with patients with moderate disability (GOS Score 4) (p<0.001) and patients with mild to no disability (GOS Score 5) (p<0.05). Similarly, expression levels of caspase-1 (p20) and NALP-1 are significantly higher in patients with severe disability (GOS Score 3) than in those with moderate disability (GOS Score 4) (p<0.001).

The results of this study show that inflammasome proteins (e.g., ASC, NALP-1, and caspase-1) are acutely elevated (e.g., within 72 hours) in the CSF of patients with TBI as compared with nontrauma controls. Elevation of these proteins likely reflects the extent of neuroinflammation, suggesting that inflammasome proteins can serve as acute biomarkers of CNS injury. These findings are clinically relevant, as CSF biomarkers are more specific indicators of neuropathology than serum biomarkers. Cerebrospinal fluid directly bathes the brain, closely reflecting the extracellular milieu and biochemical changes that are specific to the CNS. Sampling the CSF eliminates influences of multiorgan trauma or other systemic pathology represented in the serum, which is significant as patients with TBI often present with trauma to other organ systems.

The results also demonstrate that levels of inflammasome proteins are significantly higher in the CSF of patients who have died and those with severe disability than in patients with moderate to no disability, suggesting that inflammasome activation produces chronic neuroinflammation, contributing to secondary injury and poor outcome 5 months after TBT. The extent of acute elevation of these proteins can predict an unfavorable versus favorable outcome. Such markers could also direct treatment and rehabilitation efforts. The clinician would target therapies to patients identified as having a greater risk of inflammation-mediated secondary injury.

Response to treatment could be monitored by following the levels of ASC, active caspase-1, and NALP-1 in the CSF. One such treatment, therapeutic hypothermia, attenuates the endogenous inflammatory response of the CNS to TBI by decreasing cytokine production and reducing activation of astrocytes and microglia (Aibiki, et al., 1999; Goss, et al., 1995; Kumar, et al., 1997; Truettner, et al., 2005), and cortical neurons exposed to moderate hypothermia in culture show a decrease in activation of the inflammasome (Tamura et al., in press). Thus, ASC, active caspase-1, and NALP-1 can serve as objective, biochemical indicators of treatment efficacy for patients with CNS injury.

### Example 4. Hypothermia Decreases Caspase-1 Activation after Traumatic Brain Injury in Pediatric Patients

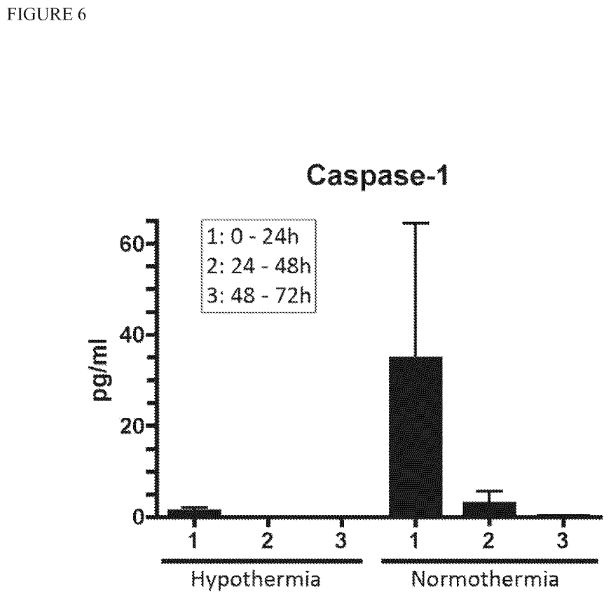

To evaluate whether inflammasome proteins, such as caspase-1, can also be used to monitor treatment efficacy in TBI patients, CSF caspase-1 levels obtained from pediatric patients who received hypothermia treatment following TBT were compared to those obtained from pediatric patients who did not receive treatment following TBI. Cerebrospinal fluid of pediatric patients (ages 0.1 to 16 years) was obtained at different times after traumatic brain injury (day 1, 2 and 3). Patients were divided into those that received hypothermia treatment and those who did not (normothermia). As shown in FIG. 6, the data indicate that within 24 hours after injury the levels of caspase-1 were lower in the hypothermia group when compared to the normothermia group. Thus, post-traumatic therapeutic hypothermia lowers the levels of caspase-1 activation after brain injury, consistent with findings that those patients that receive hypothermia treatment present better outcomes when compared to those patients who do not get the hypothermia treatment.

### Example 5. TBI IN ADULT PATIENTS

### Materials and Methods

The study was approved by the Comité Ético de las Islas Baleares (IRB protocol number 3127/15).

### Participants

Study specimens from TBI patients were acquired Son Espases University Hospital (Palma de Mallorca, Spain). The study was approved by the Comité Ético de las Islas Baleares (IRB protocol number 3127/15). Written informed consent was obtained from a family member or proxy according to the IRB (Table 3). Healthy age matched controls were acquired from BioIVT (Hicksville, NY). Informed consent was obtained from specimen donors. Control samples were obtained by donors participating in the study Prospective Collection of Samples for Research funded by SeraTrials, LLC. with IRB number 20170439. Blood samples from TBI patients used in this study were collected in the range of approximately 60 to 720 minutes after TBI with a median of 367.5 minutes (˜6 hours after TBI). Exclusion criteria in this study consisted of patients with normal findings on the CT scan on admission, patients with a major extracranial trauma (defined as extracranial Injury Severity Score >18 points), and patients with past medical history relevant to CNS pathology such as brain tumor, meningitis, cerebral vasculitis or stroke.

### Data Collection

Patients' clinical data were recorded and reviewed using the electronical medical records from the hospital (Power Chart; Millenium, 2011, Cerner Corporation, Kansas City, Missouri, USA). We collected all the variables included in the International Mission for Prognosis and Analysis of Clinical Trials in TBI (IMPACT) prognostic calculator for each patient. We also collected the GCS that first responders wrote in their prehospital report or the hospital admission GCS if the former was not available. The 6-month outcome was assessed using the extended version of the Glasgow Outcome Scale (GOSE) by a trained Neurosurgery Intensive Care Unit attending (JRP) by telephone consultation, and he was blinded to biomarker analysis.

TABLE 3
Summary of demographic data and clinical
characteristics in patients with TBI

| | TBI (N = 93) | |
|---|---|---|
| Gender (n, %) | | |
| Male | 74 | (80%) |
| Female | 19 | (20%) |
| Age (years) median (Range) | 47 | (15-83) |
| Injury Mechanism (n; %) | | |
| Fall | 52 | (56%) |
| Assault | 5 | (5%) |
| MVA | 36 | (39%) |
| Glasgow Coma Scale (n; %) | | |
| 3-8 | 37 | (40%) |
| 9-12 | 20 | (21%) |
| 13-15 | 34 | (37) |
| Undetermined | 2 | (2%) |
| Motor score (n; %) | | |
| M6 | 38 | (41%) |
| M5 | 29 | (32%) |
| M4 | 4 | (4%) |
| M3 | 2 | (2%) |
| M2 | 1 | (1%) |
| M1 | 14 | (15%) |
| Undetermined | 5 | (5%) |
| Pupillary Reactivity (n; %) | | |
| Both reactive | 78 | (84%) |
| 1 reactive | 8 | (9%) |
| None reactive | 7 | (7%) |
| Hospital length of stay (days) median (Range) | 13 | (1-149) |
| ICU length of stay (days) median (Range) | 5 | (1-90) |

TBI: Traumatic Brain injury;
MVA: motor vehicle accident;
ICU: Intensive Care Unit

### Simple Plex Assay

The serum concentration of inflammasome proteins (Caspase-1, ASC and IL-18) were measured in TBI patients and in age matched controls via Ella System (Protein System) as described in Weaver, C., Cyr, B., de Rivero Vaccari, J. C. and de Rivero Vaccari, J. P. (2020). Inflammasome Proteins as inflammatory Biomarkers of Age-Related Macular Degeneration. Transl Vis Sci Technol 9, 27. Briefly, samples were loaded as 50 iL of diluted sample into sample wells of a CART with 1 mL of washing buffer loaded separately into respective buffer wells. Assay was run using the Runner Software (version 3.5.2.20). Samples were then automatically analyzed utilizing the Simple Plex Explorer (version 3.7.2.0).

### MSD V-PLEX Inflammatory Panel

Serum levels of the inflammatory cytokines TNF-αt, IL-2, IL-4, IL-6, IL-8, IL-10, IL-13 and interferon (IFN)-γ was measured utilizing the V-PLEX Proinflammatory Panel 1 (MSD) as in Scott, X. O., Stephens, M. E., Desir, M. C., Dietrich, W. D., Keane, R. W. and de Rivero Vaccari, J. P. (2020). The Inflammasome Adaptor Protein ASC in Mild Cognitive Impairment and Alzheimer's Disease. Int J Mol Sci 21. All relevant controls, detection antibodies, standards, reagents, and dilutants were supplied by and prepared in accordance with manufacturer's instructions. Briefly, samples were diluted 2-fold prior to loading into the plate. Plate wells were washed 3 times with wash buffer prior to sample loading. Fifty pL of sample was loaded into respective plate wells and allowed to incubate for 2 hours at room temperature on a plate shaker. After incubation, plate wells were washed 3 times with wash buffer. Detection antibody was then added to plate wells and allowed to incubate for 2 hours at room temperature on a plate shaker. After antibody incubation, plate wells were washed 3 times with wash buffer. 2X Read buffer was then added to each well and the plate was analyzed utilizing the MESO QuickPlex SQ120 (MSD) and DISCOVERY WORKBENCH software (version 4.0.12).

### Statistical & Biomarker Analysis

Simple Plex and V-PLEX data from TBI and control samples were analyzed utilizing Prism 9 software (GraphPad). Outliers were removed prior to further statistical analyses using the Robust regression and Outlier removal (ROUT) method with a Q set to 1%. Descriptive statistics were run, and normality was determined by the Shapiro Wilk-Test or the D'Agostino & Pearson Test. Non-parametric data were analyzed using a two-tailed Mann-Whitney test and parametric data were analyzed using a two-tailed t-test. P-value of significance was set to p<0.05.

Receiver operating characteristics (ROC) were calculated to obtain the area under the curve (AUC) in order to obtain cut-off points and the respective specificity, sensitivity and likelihood ratio. The cut-off point for each analyte was chosen based on the highest likelihood ratio in the sensitivity vs. 1-specificity plot favoring a higher sensitivity than specificity values, to obtain assays with higher likelihood of reliability for each analyte. Positive and negative predictive values were also calculated along with overall assay accuracy.

Comparison of ROC curves between inflammatory biomarkers was carried out as described in Hanley, J. A. and McNeil, B. J. (1983). A method of comparing the areas under receiver operating characteristic curves derived from the same cases. Radiology 148, 839-843 using the following formula to obtain a critical ratio Z:

$$z = \frac{( {A}_{1} - {A}_{2} )}{\sqrt{{SE}_{1}^{2} + {SE}_{2}^{2} - 2 {rSE}_{1} {SE}_{2}}}$$

The p-value was determined using the following formula using Microsoft Excel (version 16.57): =2*(1-NORMSDIST(z)) A Pearson correlation was carried out to obtain r in order to calculate the z-score to allow for comparison of ROC curves between analytes obtained from the same samples.

Linear regression analysis to explain the GCS and the GOS-E were fit using all the inflammatory proteins analyzed in this study through a stepwise approach based on the lowest Akaike information criterion (AIC) using RStudio/RMarkdown (Version 1.2.5033) and then fitted to obtain the estimate, standard error and p-values for each predictor and the intercept. The Bayesian information criterion (BIC), residuals, root mean-square error (RMSE), mean of residuals, confidence intervals, and the autocorrelation using the Durbin Watson (DW) statistic were then calculated for the best fit model. The Durbin Watson (DW) statistic was used to test for autocorrelation. After identifying a best fit model, data points underwent logarithmic transformation to normalize the distribution of the data. An adjusted r-squared value was obtained to determine the approximate contribution of these three proteins to either the GCS or the GOS-E. The final models were then further evaluated by residual analysis with and without logarithmic transformation.

### Results

### Inflammasome Proteins and Inflammatory Cytokines are Elevated in TBI Patients

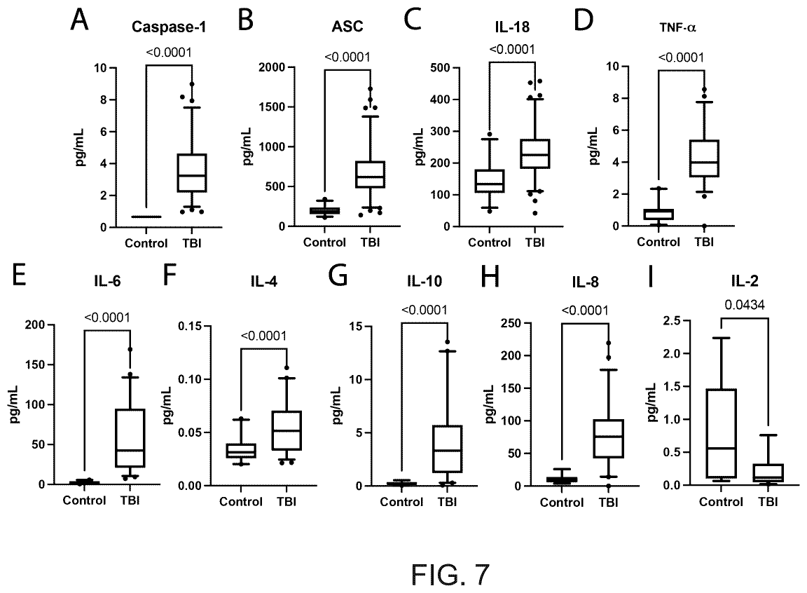

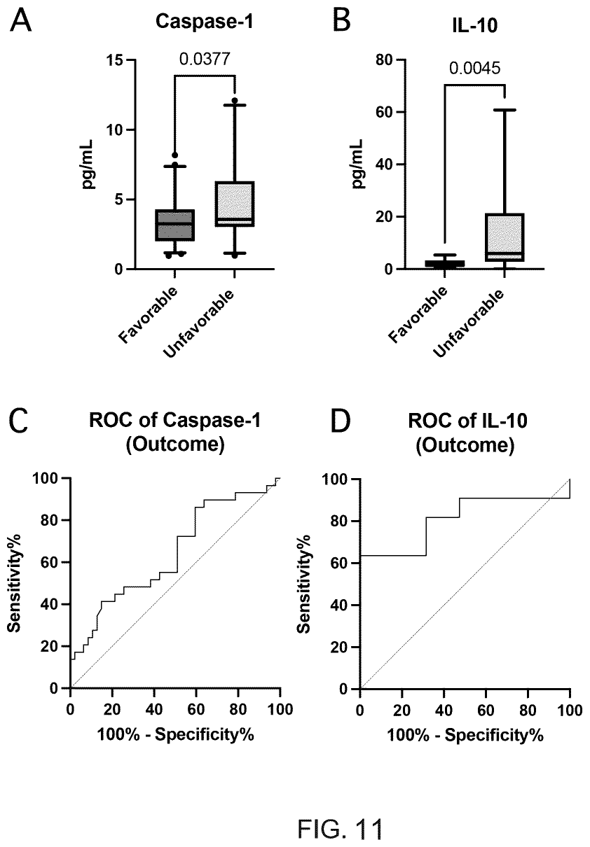

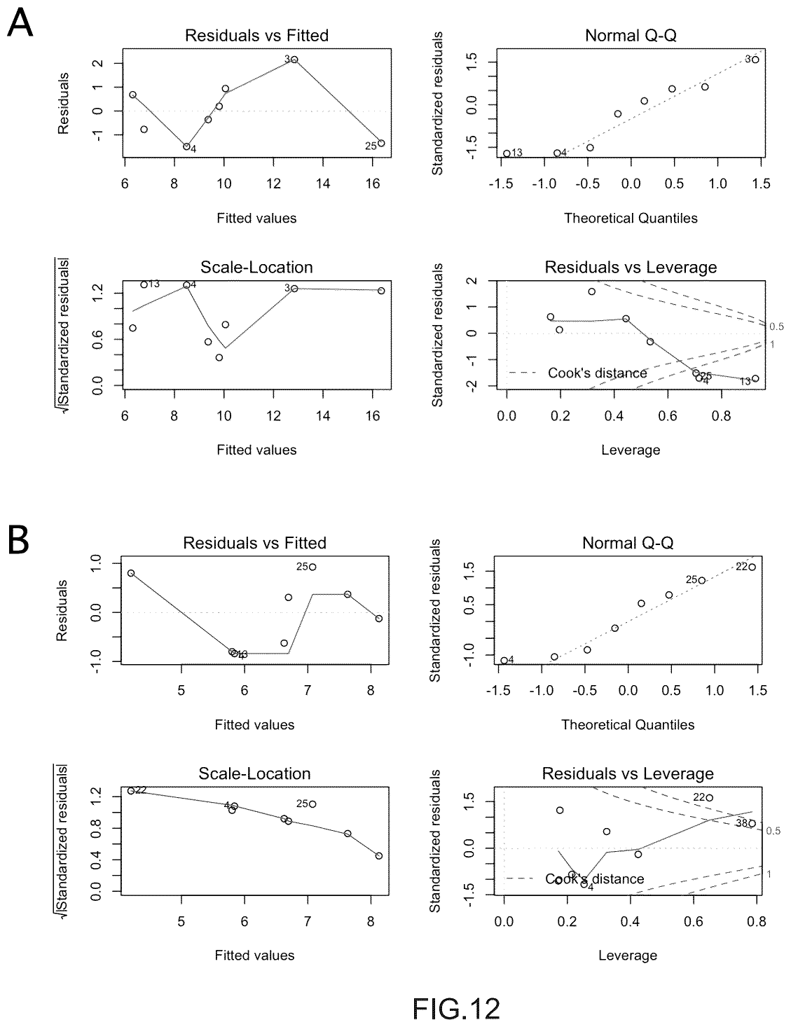

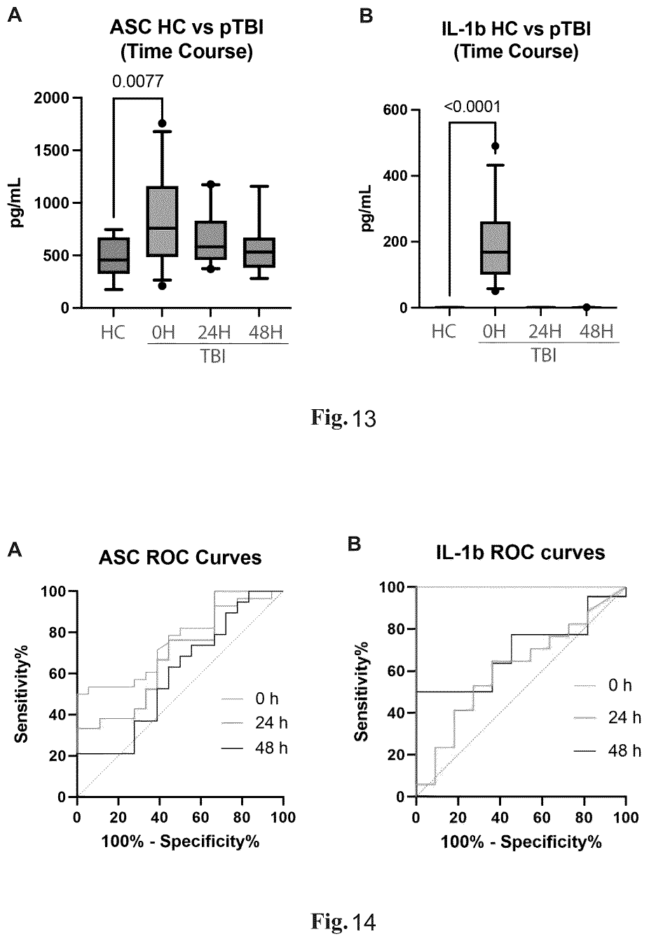

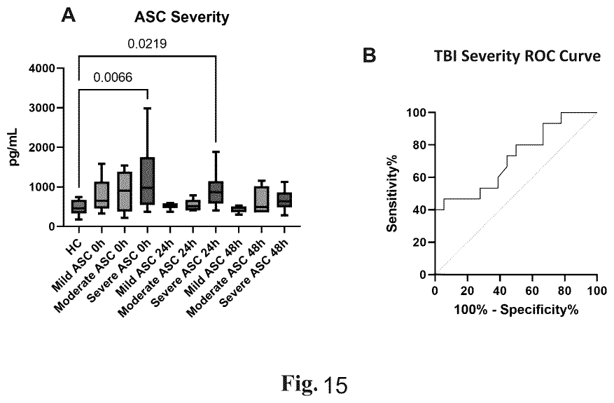

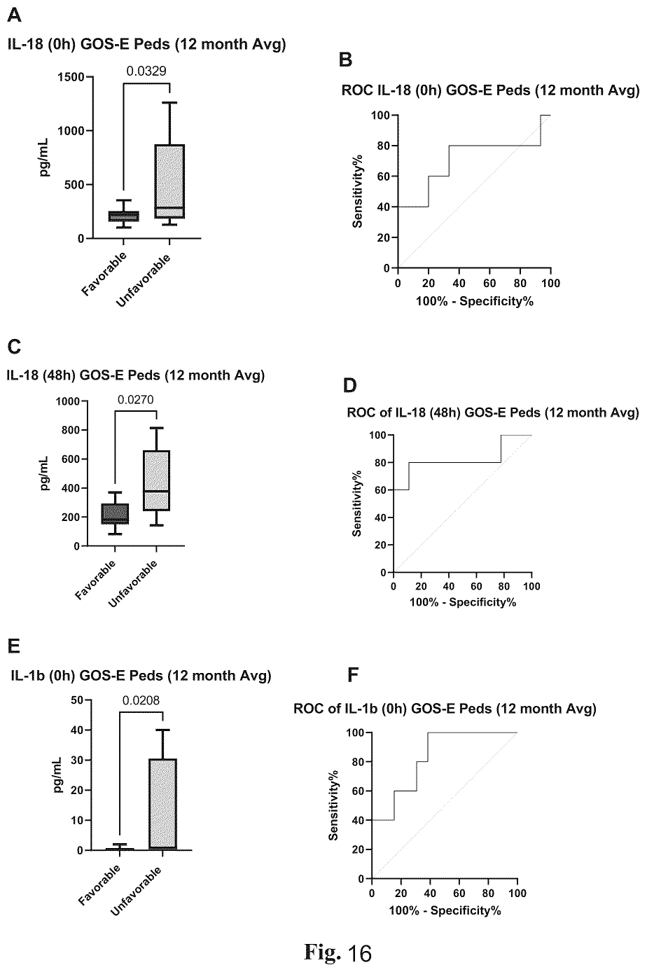

Increased inflammatory activity through inflammasome and cytokine signaling has been previously reported in animal and human TBI studies. In order to determine which inflammatory proteins were elevated in this cohort of human TBI patients, we analyzed the levels of inflammasome proteins caspase-1 (FIG. 7A), ASC (FIG. 7B) and IL-18 (FIG. 7C), and the inflammatory cytokines TNF-α (FIG. 7D), IL-6 (FIG. 7E), IL-4 (FIG. 7F), IL-10 (FIG. 7G), IL-8 (FIG. 7H) and IL-2 (FIG. 71) in the blood serum of these patients and compared them to age matched healthy controls. TBI patients had significantly elevated levels of inflammasome signaling proteins caspase-1 (FIG. 7A), ASC (FIG. 7B) and IL-18 (FIG. 7C), as well as significantly elevated levels of cytokines TNF-α (FIG. 7D), IL-6 (FIG. 7E), IL-4 (FIG. 7F), IL-10 (FIG. 7G) and IL-8 (FIG. 7H). In contrast, the levels of IL-2 were higher in the serum of healthy uninjured controls when compared to the serum of TBI patients (FIG. 11). Therefore, these results indicate that TBI patients have sustained an acute increase in inflammatory activity after TBI.

### Inflammatory Biomarkers of TBI

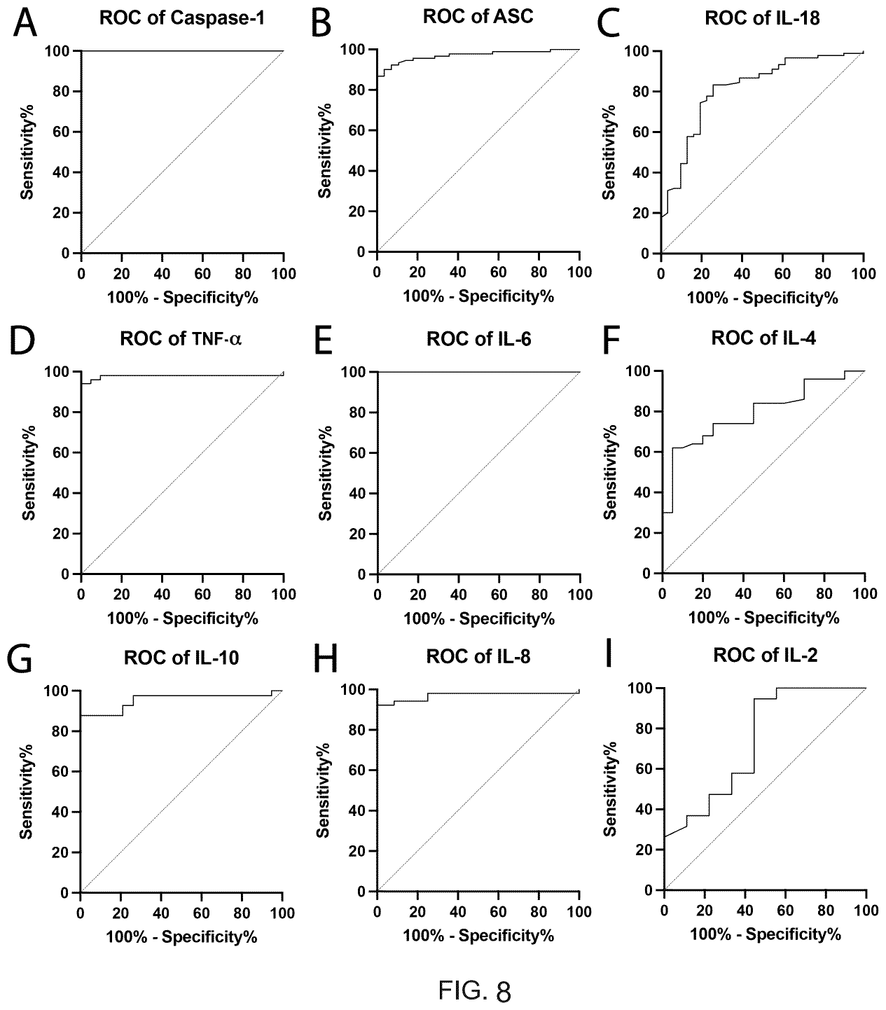

Previous studies have shown that inflammasome proteins are potentially promising biomarkers for determining TBI pathological outcomes. In order to determine the biomarker reliability of the inflammasome proteins inflammasome proteins caspase-1 (FIG. 8A), ASC (FIG. 8B) and IL-18 (FIG. 8C), and the inflammatory cytokines TNF-α (FIG. 8D), IL-6 (FIG. 8E), IL-4 (FIG. 8F), IL-10 (FIG. 8G), IL-8 (FIG. 8H) and IL-2 (FIG. 81) in the context of TBI, we plotted the ROC curve for each protein (FIG. 8). Of the inflammatory cytokines examined, IL-6 (FIG. 8E) had the highest AUC 1.0 (Table 3) with a sensitivity of 100% and a specificity of 100% (Table 5). TNF-α had an AUC of 0.98 AUC (96% sensitivity, 95% specificity), IL-10 and IL-8 also presented high AUC values (0.97 and 0.95, respectively), whereas IL-4 had an AUC of 0.79 (74% sensitivity, 75% specificity) and IL-2 an AUC of 0.74 (95% sensitivity, 56% specificity).

Of the inflammasome proteins examined, caspase-1 (FIG. 2A) had the highest AUC at 1.0 (Table 4) with a sensitivity of 100% and a specificity of 100%, followed by ASC with an AUC of 0.97 with a sensitivity of 92% and a specificity of 93% (Table 3). IL-18 presented an AUC of 0.81 (sensitivity of 83%, specificity of 74%). These results suggest that caspase-1, ASC, IL-18, TNF-α, IL-6 IL-8 and IL-10 are reliable biomarkers of TBI with AUC values above 0.80.

TABLE 4
ROC Analysis

| Biomarker | Area | Std. Error | 95% C.I. | p-value |
|---|---|---|---|---|
| Caspase-1 | 1.0 | 0 | 1.0 to 1.0 | <0.0001 |
| ASC | 0.97 | 0.01384 | 0.9428 to 0.9971 | <0.0001 |
| IL-18 | 0.8143 | 0.04538 | 0.7254 to 0.9033 | <0.0001 |
| TNF-α | 0.9776 | 0.01963 | 0.9391 to 1.000 | <0.0001 |
| IL-6 | 1.0 | 0 | 1.0 to 1.0 | <0.0001 |
| IL-4 | 0.7945 | 0.05424 | 0.6882 to 0.9008 | 0.0001 |
| IL-10 | 0.9538 | 0.02697 | 0.9009 to 1.0 | <0.0001 |
| IL-8 | 0.9696 | 0.2115 | 0.9281 to 1.0 | <0.0001 |
| IL-2 | 0.7398 | 0.1084 | 0.5274 to 0.9522 | 0.0437 |

TABLE 5
Cut-off point in serum of TBI patients

| | Cut-off point | Sensitivity | Specificity | | PPV | NPV | Accuracy |
|---|---|---|---|---|---|---|---|
| Biomarker | (pg/ml) | (%) | (%) | LR | (%) | (%) | (%) |
| Caspase-1 | >0.8150 | 100 | 100 | | 100 | 100 | 100 |
| ASC | >284 | 92 | 93 | 12.92 | 98 | 79 | 92 |
| IL-18 | >156 | 83 | 74 | 3.229 | 90 | 61 | 81 |
| TNF-α | >2.202 | 96 | 95 | 20.18 | 98 | 91 | 96 |
| IL-6 | >6.443 | 100 | 100 | | 100 | 100 | 100 |
| IL-4 | >0.03868 | 74 | 75 | 2.96 | 88 | 54 | 74 |
| IL-10 | >0.6527 | 88 | 100 | | 100 | 79 | 92 |
| IL-8 | >29.18 | 92 | 100 | | 100 | 75 | 94 |
| IL-2 | <0.5145 | 95 | 56 | 2.132 | 82 | 83 | 82 |

Comparison between ROC curves for identified inflammatory biomarkers

To compare the ROC curves for caspase-1, ASC, IL-18, TNF-α, IL-6, IL-4, IL-10, IL-8 and IL-2, a Pearson correlation coefficient was first obtained from a correlation matrix (FIG. 3A). Accordingly, of all the correlations run, the highest correlation was found between ASC and caspase-1 with a coefficient of correlation of 0.8, followed by a correlation of 0.58 between IL-6 and TNF-α, 0.57 between IL-6 and IL-8 and 0.53 between IL-6 and IL-4. After finding the coefficient of correlation, the ROC curves were compared according to the formula:

$$z = \frac{( {A}_{1} - {A}_{2} )}{\sqrt{{SE}_{1}^{2} + {SE}_{2}^{2} - 2 {rSE}_{1} {SE}_{2}}}$$

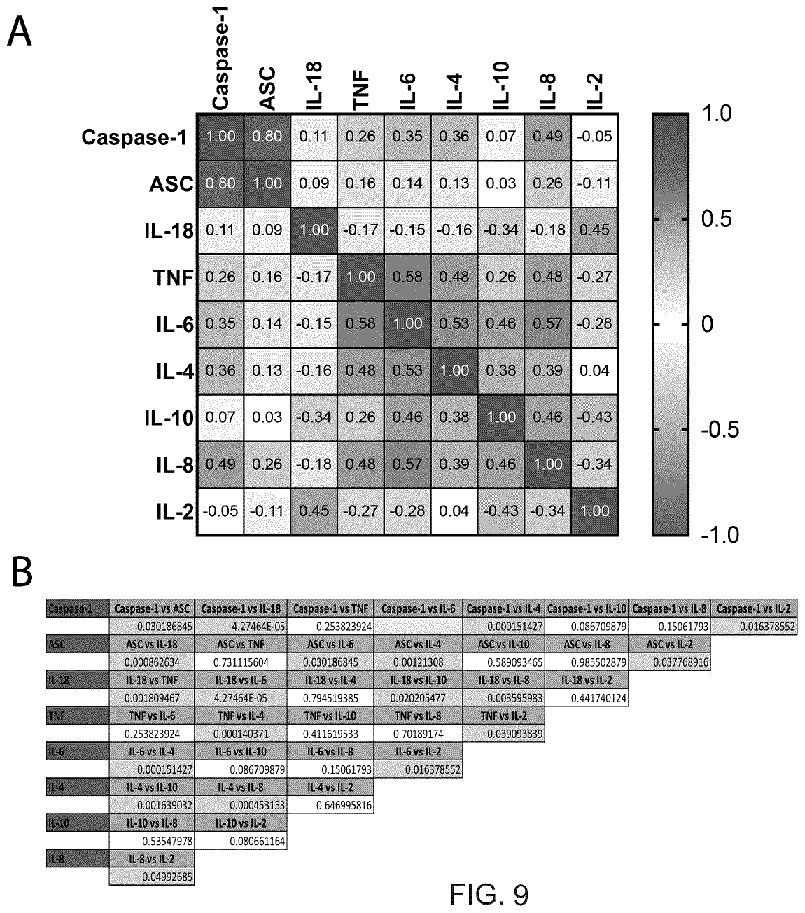

ROC curve comparison analysis indicated that the ROC between caspase-1 and ASC (p=0.03), caspase-1 and IL-18 (p=4.27E-05), caspase-1 and IL-4 (p=0.0001) as well as caspase-1 and IL-2 (p=0.01) were significantly different from each other (FIG. 9B). Similarly, the ROC between ASC and other analytes differed from IL-18 (p=0.0009), IL-6 (p=0.03), IL-4 (p=0.001) and IL-2 (p=0.04). For IL-18, it also differed from TNF-α (p=0.002), IL-6 (p=4.27E-05), IL-10 (p=0.02) and IL-8 (p=0.004). For TNF-α, the ROC curves also differed with IL-4 (p=0.0001) and IL-2 (p=0.04). For IL-6, the ROC curves differed with IL-4 (p=0.0002) and IL-2 (p=0.02). For IL-4, IL10 (p=0.002) and IL-18 (p=0.0005), and for IL-8 the ROC curve also differed with that of IL-2 (p=0.05).

Taken together, these analysis highlights caspase-1 and IL-6 as useful inflammatory biomarkers superior to all other biomarkers examined in this study; however, caspase-1 and IL-6 were not different from each other (FIG. 9B). Similarly. ASC and TNF-α were not different from each other but ASC was superior to IL-18 and IL-4. Similarly, IL-10 and IL-8 were not different from each other, and IL-8 was superior to IL-2.

### Inflammatory Biomarkers of Injury Severity

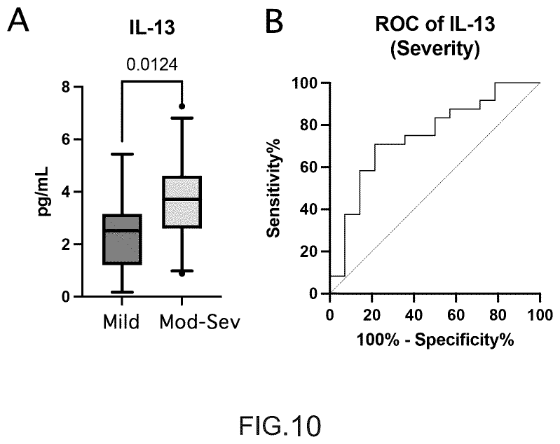

We then screened all inflammatory biomarkers analyzed to determine whether there was a difference in the levels of these proteins between patients that presented mild TBI and those with moderate to severe TBI as determined by the GCS. Patients with mild TBI were those who presented a GCS between 13 and 15; whereas patients with a GCS between 3 and 12 were grouped in the moderate to severe cohort. Of all the analytes measured in this study, IL-13 was the only protein to be elevated in the moderate to severe group when compared to patients in the mild TBI group (FIG. 10A). Moreover, we then calculated the ROC curve for IL-13 and found that the AUC for IL-13 in regard to its ability to discriminate between mild and moderate to severe TBI was 0.75 (FIG. 10B) with a 95% confidence interval between 0.5815 to 0.9126 and an SEM of 0.085 (p=0.01). Furthermore, with a cut-off point of 3.12 pg/mL, the sensitivity and specificity of IL-13 were 71% and 79%, respectively. This resulted in a PPV of 85% and a NPV of 61% with an accuracy of 74%.

Then a multivariate linear regression model was fit using a stepwise approach to predict what inflammatory biomarkers contribute to the GCS. Accordingly, using as predictors the inflammatory proteins caspase-1, ASC, IL-18, TNF-α., IL-2, IL-4, IL-6, IL-8, IL-10, IL-12, IL-13 and IFN-t.

The best model was chosen based on the AIC (14.37) by the stepwise method, and then the estimate (coefficients), standard error and p-values for each predictor and intercept (slope), as well as the BIC (35.52), residuals (FIG. 6A), RMSE (1.65), mean of residuals (−6.25E-17), confidence intervals, and the DW autocorrelation test for the best fit model were calculated. An adjusted R² value (0.78) was obtained for the model to determine the approximate contribution of the fitted model to the GCS (Table 6). Thus, based on this model, we determined that the GCS score is contributed in part by IL-12, IL-13 and the log(IL-12), considering an adjusted R² of 0.78 and a p-value of 0.03, consistent with our findings of IL-13 as a biomarker of injury severity after TBI based on the GCS.

TABLE 6
Linear Regression Model to predict GCS

| GCS | Estimate | Std. Error | p-value | Confidence Interval |
|---|---|---|---|---|
| Intercept | 21.245347 | 2.221427 | 0.000668 | 15.07767794 to 27.413016974 |
| IL-13 | −1.519195 | 0.332622 | 0.010282 | −2.44270084 to −0.595688402 |
| IL-12 | −0.016000 | 0.004802 | 0.029063 | −0.02933364 to −0.002666043 |
| LOG(IL-2) | 2.095605 | 0.670269 | 0.035304 | 0.23463864 to 3.956571427 |
| Adjusted R² | 0.7798 | | | |
| BIC | 35.51754 | | | |
| RMSE | 1.644861 | | | |
| Mean of Residuals | −6.25E−17 | | | |
| DW Statistic | | | | |

| | |
|---|---|
| Rho ! = 0 | p-value = 0.812 |
| rho < 0 | p-value = 0.443 |
| rho > 0 | p-value = 0.561 |

GCS = 21.25 − 1.52(IL-13) − 0.02(IL-12) + 2.10 * LOG(IL-12)

### Inflammatory Biomarkers of Outcome

To then determine the contribution of inflammatory proteins to outcomes according to the GOS-E, we first divided the outcomes as favorable and unfavorable and then determined if there was a statistically significant difference between the levels of the inflammatory proteins caspase-1, ASC, IL-18, TNF-α, IL-2, IL-4, IL-6, IL-8, IL-10, IL-12, IL-13 and IFN-γ in regard to favorable (GOSE scores of 5-8) v's. unfavorable (GOSE scores of 1-4) outcomes. Of the protein analyzed, caspase-1 (FIG. 11A) and IL-10 (FIG. 11B) were the two proteins that were significantly elevated in those patients that presented unfavorable outcomes.

Following identification of caspase-1 and IL-10 as proteins that were elevated in patients with unfavorable outcomes, we aimed to identify whether these two analytes are good biomarkers of outcomes in TBI patients. Accordingly, the ROC curves of caspase-1 (FIG. 11C) and IL-10 (FIG. 11D) were 0.64 (p=0.03) and 0.81 (p=0.006), respectively. The SEM for the ROC of caspase-1 was 0.07 with a 95% confidence interval between 0.5127 and 0.7720. For p-10, the SEM was 0.097 with a 95% confidence interval between 0.6182 and 0.9990. Moreover, with a cut-off point of 3.33 pg/mL, and a likelihood ratio of 1.3, the sensitivity and specificity of caspase-1 were 55 and 57%, respectively, with a PPV of 68% and a NPV of 44% with an accuracy of 56%. In contrast, for IL-10, with a cut-off point of 5.55 pg/mL, the sensitivity and specificity were 64 and 100%, respectively, with a PPV of 100% and a NPV of 61% with an accuracy of 77%.

We then fitted a multivariate linear regression model using caspase-1 and IL-10 as the predictors to explain the GOS-E. After analyzing residuals (FIG. 11B) and adding a logarithmic transformation for caspase-1 levels, the adjusted R² value (0.65) wasobtained forthe model to determinethe approximate contribution of I-10 and caspase-1 to the GOS-E (p=0.03) (Table 7).

TABLE 7
Linear Regression Model to predict GOS-E

| GCS | Estimate | Std. Error | p-value | Confidence Interval |
|---|---|---|---|---|
| Intercept | 9.1238 | 0.7938 | 8.74e−05 | 7.0832670 to 11.1643303 |
| IL-10 | 0.2483 | 0.1416 | 0.1398 | −0.1156099 to 0.6122821 |
| LOG(Caspase-1) | −2.5997 | 0.6700 | 0.0116 | −4.3220676 −0.8772845 |
| Adjusted R² | 0.6524 | | | |
| BIC | 24.35273 | | | |
| RMSE | 0.8338086 | | | |
| Mean of Residuals | 1.39e−17 | | | |
| DW Statistic | | | | |

| | |
|---|---|
| rho != 0 | p-value = 0.574 |
| rho < 0 | p-value = 0.716 |
| rho > 0 | p-value = 0.316 |

GOS-E = 9.12 + 0.25(IL-10) − 2.60 * LOG(caspase-1)

### DISCUSSION

TBI is a significant medical condition which represents a major source of potential disability and economic strain. The effects of TBI are chronic with increased inflammatory activity and long-term learning and memory loss. In order to improve the care of patients with TBI, non-invasive diagnostic tools are needed. To this end, the serum of TBI patients is an ideal source to measure the diagnostic and prognostic potential of signaling proteins that can provide clinicians with the biochemical status of patients as it pertains to neuronal damage, astrocyte activation, and the inflammatory response involving microglial neutrophils and other inflammatory cells. To date, UCH-L1 has been approved by the FDA as a biomarker of neuronal damage after TBI, whereas GFAP has been approved as a biomarker of reactive astrogliosis. However, to date no inflammatory marker in serum has been approved for the care of patients with TBI.

Due to their higher sensitivity and consistency across assays, modern tools for biomarker analysis provide the ability to identify biomarkers in serum that normally would have only been detected in the CSF. In this study we used two technologies to measure inflammatory biomarkers in the serum of patients with TBI. One tool uses electrochemiluminescence (MESO QuickPlex SQ120, MSD) and the other one, microfluidics (Ella, Protein Simple). Using this highly sensitive technology, in this study we were able to examine the expression levels of inflammatory proteins including inflammasome signaling proteins and inflammatory cytokines in the blood serum of patients after TBI and investigated their potential as reliable diagnostic and prognostic biomarkers of TBI.

Previous studies have measured a variety of inflammatory proteins in healthy individuals and TBI patients. However, many of these studies did not evaluate the actual biomarker characteristics of such proteins. In order to determine the potential biomarker role that an analyte might have, it is not sufficient to only measure levels of proteins in control and TBI groups. For biomarker analyses, the ROC curve should be calculated in order to obtain the AUC by plotting the sensitivity in the y-axis and 1-specificity in the x-axis for each analyte. In addition, cut-off points should be identified with their respective sensitivity and specificity. Therefore, here we used a systematic approach to identify reliable diagnostic biomarkers capable of providing clinical information regarding the inflammatory status of patients after TBI using modern approaches for protein analysis. Accordingly, we first measured the levels of the candidate biomarkers to determine the protein levels of the inflammasome signaling proteins caspase-1, ASC and IL-18 and the inflammatory cytokines TNF-α, IL-2, IL-4, IL-6, IL-8 and IL-10 in the serum of healthy uninjured controls and TBI patients collected within the first day after TBI (range of approximately 60 to 720 minutes after TBI with a median of 367.5 minutes (˜6 hours after TBI)).

Our first analysis revealed that the inflammasome proteins caspase-1, ASC and IL-18 were elevated after TBI consistent with previous studies showing similar results in humans and rodents. Interestingly, the lower limit of quantitation (LLOQ) for caspase-1 was 0.66, and all the uninjured controls presented levels below the detection level of the assay, whereas the TBI group presented protein levels with a range between 3.224 and 4.051 pg/mL. Similarly, protein levels of the inflammatory cytokines TNF-α, IL-4, IL-6, IL8 and IL-10 were significantly elevated in the serum of TBI patients compared to controls, indicating a balance between pro-inflammatory (TNF-α, IL-6) and anti-inflammatory (IL-4, IL-10) cytokines at play acutely after TBI. In contrast, IL-2 levels were elevated in the serum of healthy controls when compared to TBI patients, suggesting a loss of this cytokine acutely after injury and the potential loss of a mechanism responsible for keeping the pro-inflammatory environment at check. IL-2 is a growth factor that is secreted by activated T cells and plays a role in the generation of immunity through the proliferation and differentiation of B cells and T cells, and the amplification of NK cell activity. Our findings are consistent with the pro-inflammatory role that the absence of IL-2 results in and is similar to what is seen in like CNS injury, such as Traumatic Brain Injury (TBI). For instance, serum levels of IL-2 have been shown to be decreased in patients after ischemic stroke³². Additionally, IL-2 or IL-2R elimination has been shown to result in systemic autoimmunity.

Since simple measurement of protein levels are not sufficient for biomarker analysis, we then aimed to determine the ROC curve for all the analytes in which there was a statistically significant difference between the control and the TBI group. It has been suggested that an AUC between 0.9 to 1.0 corresponds to an excellent biomarker; from 0.8 to 0.9, a good biomarker; from 0.7 to 0.8, a fair biomarker; from 0.6 to 0.7, poor and from 0.5 to 0.6, a failed analyte.³⁵ In this study, we found that caspase-1 and IL-6 with an AUC of 1.0 were the best inflammatory biomarkers of those examined in this study, followed by TNF-α and ASC with an AUC of 0.98 and 0.97, respectively, and IL-8 and IL-10 with an AUC of 0.97 and 0.96, respectively. Furthermore, the most sensitive inflammatory biomarkers were caspase-1 and IL-6, followed by TNF-α, IL-2, ASC and IL-8, whereas the most specific biomarkers were caspase-1, IL-6, IL-8. IL-10, TNF-α, ASC. When looking at the accuracy of each biomarker, caspase-1 and IL-6 presented an accuracy of 100%, followed by TNF-α with 96%, IL-8, 94% and ASC and IL-2 with an accuracy of 92%. Taken together these data suggest that caspase-1, ASC, TNF-α, IL-6, IL-10 and IL-8 are the most reliable diagnostic inflammatory biomarkers of TBI, among those studied. However, when there are several biomarkers that have similar AUC values, it is important to determine if the ROC curves differ among the different biomarkers. Thus, following determination of the ROC curves for each biomarker, we then compared the ROC for each of them, and identified significant differences between different biomarkers despite many of them having high AUC values. For instance, we found significant differences between caspase-1 and ASC. However, the ROC for caspase-1 and IL-6 were not significantly different, indicating that both of these analytes have the same biomarker potential based on their respective biomarker characteristics, yet this is not to say that each of these biomarkers does not provide different information pertaining the acute inflammatory response after TBI. Similarly, the ROC curve for ASC and TNF-were not found to be significantly different either, and the AUC of IL-6 was found to be superior to that of ASC, consistent with the significance found between caspase-1 and ASC. Overall, the findings of this study highlight the importance of caspase-1, ASC, IL-6 and TNF as pro-inflammatory biomarkers of the acute response after TBI, whereas IL-10 was the best anti-inflammatory biomarker.

In this study, we dichotomized the GOS-E into favorable and unfavorable outcomes to identify whether any of the inflammatory proteins analyzed in this study were significantly different between patients with different outcomes after TBI, and then test whether these proteins were effective prognostic inflammatory biomarkers of TBI. Accordingly, we found that caspase-1 and IL-10 were elevated in patients with unfavorable outcomes when compared to those with favorable outcomes. IL-10 is secreted by numerous cells of the CNS after injury and has been shown to play a protective role by reducing cytokine activity, proinflammatory activity, and apoptosis. Additionally, IL-10 inhibits IL-2 activity, and has been shown in numerous studies to be increased after stroke or TBI. Interestingly, although IL-10 has a pro-survival purpose, increased IL-10 expression after injury has been associated with worsened pathological outcomes with higher expression associated with increased chance for mortality. Our results reinforce those previous observations and further suggest an interplay between pro-inflammatory (caspase-1) and anti-inflammatory (IL-10) proteins towards the contribution to long-term outcomes in patients with TBI. Biomarker analysis indicated that the AUC for caspase-1 was 0.64 and the AUC for IL-10 was 0.81, suggesting that among all the inflammatory proteins that we have analyzed in this study, IL-10 was the better biomarker of long-term outcome with a cut-off point of 5.55 pg/mL The addition of caspase-1 and IL-10 as prognostic biomarkers further implicates the role that inflammation plays on patient outcomes. Previous studies have shown that serum levels of TNF-α remain elevated for at least one-year post-injury. Serum levels of IL-6 have been shown to be more elevated in more severe cases of TBI, and that these elevated IL-6 levels were associated with worsened outcomes. IL-8 is similar in that studies have also shown that increased IL-8 expression in serum or CSF of TBI patients is associated with increased chance for mortality and overall worsened pathological outcomes. This is thought to be due to its chemoattractant properties in which it is able to recruit and activate monocytes to the site of injury, increasing the overall inflammatory response after TBI or stroke. IL-4 however seems more ambiguous, with one study showing that administration of IL-4 after injury may offer some form of protection from the effects of TBI.

When determining whether any of the inflammatory proteins analyzed in this study were significantly different between patients with mild TBI and moderate to severe TBI, we found that IL-13 was elevated in patients with moderate to severe TBI as determined by the GCS. This is an interesting observation, as previous studies have reported differing observations of IL-13 expression after TBI. The exact effect of IL-13 on pathology is still up for debate, though some studies have suggested that IL-13 plays a neuroprotective role in that it reduces inflammatory activity, reduces axonal loss, and mediates microglia polarization encouraging the adoption of the anti-inflammatory phenotype, and that treatment with IL-13 improves pathological outcomes in a murine model of TBI. Furthermore, IL-13 has been shown to have shared functionality with IL-4, and has also been shown to work with IL-2 to promote IFN production. Since there was a statistically significant difference for this analyte, we then determined whether IL-13 were a good biomarker of TBI severity. Our findings indicate that with an AUC of 0.75 and a cut-off point of 3.12 pg/mL, with a sensitivity of 71% and a of specificity 79%, IL-13 is a fair biomarker of TBI injury severity. Moreover, a multivariate linear regression model consisting of IL13, IL-2 and IL-12 indicated that combined, these 3 biomarkers contribute to the GCS with an adjusted R² of 0.78; thus highlighting the importance of IL-13 and a key biomarker of injury severity.

Therefore, ROC analysis revealed that these proteins were reliable diagnostic and prognostic biomarkers of TBI. These finding support our previous work that showed that serum levels of ASC, and CSF levels of ASC and IL-18 were reliable biomarkers of TBI, and that increased ASC levels predicted less favorable patient outcomes, and it was a reliable predictor of overall patient prognosis. Furthermore, we have recently shown that caspase-1, a key inflammasome component, correlates with increased intracranial pressure and poor outcomes in TBI patients.

In addition to TBI, we have previously shown that inflammasome proteins are reliable biomarkers of the inflammatory response in several conditions such as stroke, Alzheimer's disease, multiple sclerosis, age-related macular degeneration, psoriasis and non-alcoholic steatohepatitis, indicating that the inflammasome plays a major role in the pathophysiology of a variety of diseases affecting the CNS and the periphery. Moreover, those findings highlight the usefulness of inflammasome signaling proteins as biomarkers of injury and disease.

In conclusion, here we provide a systematic approach for inflammatory biomarker identification that includes 1) measuring the levels of inflammatory problems in the serum of affected and unaffected individuals to determine if there are statistical differences between groups. 2) Determining the diagnostic biomarker characteristics (AUC, sensitivity, specificity, likelihood ratio, accuracy, PPV and NPV) of each inflammatory protein or analyte that was statistically significant when comparing the levels between affected and unaffected individuals. 3) Comparing the ROC among the different biomarkers to identify potential biomarker differences between groups. 4) Dichotomize the GCS into mild and moderate to severe outcomes to determine if there are inflammatory biomarkers that meet the criteria as useful biomarkers of injury severity and 5) Dichotomize the GOS-E into favorable and unfavorable outcomes to determine if there are inflammatory biomarkers that meet the criteria as useful biomarkers of long-term outcomes. Taken together, in this study we have identified the caspase-1, ASC, IL-18, TNF-α, IL-2, IL-4, IL-6, IL-8, IL-10 and IL-12 as surrogate biomarkers in serum of the inflammatory response acutely after TBI. Thus, inflammatory biomarkers when combined with GFAP and UCH-L1, they offer the potential for clinicians to better understand not only the overall scope of injury but also a probable prognosis and potential for disability considering a variety of mechanisms contributing to the TBI pathology including neuronal damage (UCH-L1), reactive astrogliosis (GFAP) and inflammation (caspase-1, ASC, TNF-α and IL-6).

### Example 6. TBI in Pediatric Patients

### Materials and Methods

### Participants

Patients aged 0-18 years admitted to the University of Florida (UF) or transferred from an outside hospital to the Pediatric Intensive Care Unit (PICU) between February 2017-2020 with the diagnosis of TBI (GCS 3-15) that warranted brain imaging studies within 24 hours of injury were eligible to participate and were enrolled prospectively. Patients with evidence of psychiatric disorders were excluded. Common data elements for Pediatric Traumatic Brain Injury (pTBI), demographic information, and detailed clinical assessments were obtained, including a total of seven days of relevant critical care data points. Long term outcomes were measured by the Glasgow Outcome Coma Scale Extended for Pediatrics (GOS-E Peds) at 6, 9 and 12 months post injury. The UF Institutional Review Board 01 (IRB201600237) approved this study with a modification of informed consent, allowing for participants or their authorized representatives to provide written informed consent within 72 hours of admission. Healthy controls (HC) were recruited in the Emergency Department. These HC were ineligible for enrollment if they had a history of TBI or were admitted with a traumatic injury or acute neurological process. Timed blood samples were collected for genetic and proteomic analysis from all participants at enrollment, 24, and 48 hours post injury.

The TBI-Common Data Elements (TBI-CDE) Biospecimens and Biomarkers Working Group Consensus guidelines for plasma preparation were followed.² Blood sampling took place by venipuncture if required for routine clinical care, otherwise blood was collected via a catheter that was already in place as part of clinical care. A total of 2 cc/kg, up to max 5 ml (age 0-4 years); 10 ml (age 5-18 years). Blood samples were drawn into red top SST BD Vacutainer® Plus tubes. The analysis of this archive samples transferred from the University of Florida to the University of Miami was approved under the auspices of the University of Miami IRB (20210352: pTBIB).

### Data Collection

Samples sat upright for 30 minutes at room temperature and were then subsequently centrifuged at 4,000 rpm for 10 minutes. The cleared serum (supernatant) was pipetted and stored in aliquots of 0.5 ml. Samples were stored within 2 hours of blood draw in freezers at or below −80° C. in a biorepository at the UF McKnight Brain Institute at the University of Florida and later transferred to the University of Miami. All samples were stored in a de-identified manner with a unique study number specific to the participant.

### Simple Plex Assay

The serum concentration of inflammasome proteins (Caspase-1, ASC and IL-18) were measured in TBI patients and in age matched controls via Ella System (Protein System) as described in Adelson PD, Pineda J, Bell MJ, et al: Common data elements for pediatric traumatic brain injury: recommendations from the working group on demographics and clinical assessment. J Neurotrauma 2012; 29(4), 639-653. Briefly, samples were loaded as 50 mL of diluted sample into sample wells of a CART with 1 ml of washing buffer loaded separately into respective buffer wells. Assay was run using the Runner Software (version 3.5.2.20). Samples were then automatically analyzed utilizing the Simple Plex Explorer (version 3.7.2.0).

### Outcome Measures

The GOS-E Peds was used as the primary tool to quantify long term outcomes following TBI. The GOS-E Peds is a validated clinical scale used to quantify rehabilitative success and identify permanent disability in a patient recovering from TBI. Measurements are designed to screen for onset of new cognitive or behavioral changes, including sleep disturbances, social disruptions, and learning impairments. The GOS-E Peds was conducted at subsequent pediatric neurology follow up visits or by phone if the patient was not scheduled for a visit or was lost to follow up at TBI clinic. All surveys were administered by a trained research associate and completed directly by the patient's caregiver.

### Statistical & Biomarker Analysis

Simple Plex and V-PLEX data from TBI and control samples were analyzed utilizing Prism 9 software (GraphPad). Outliers were removed prior to further statistical analyses using the Robust regression and Outlier removal (ROUT) method with a Q set to 1%. Descriptive statistics were run, and normality was determined by the Shapiro Wilk-Test or the D'Agostino & Pearson Test. Non-parametric data were analyzed using a two-tailed Mann-Whitney test and parametric data were analyzed using a two-tailed t-test. P-value of significance was set to p<0.05.

Receiver operating characteristics (ROC) were calculated to obtain the area under the curve (AUC) in order to obtain cut-off points and the respective specificity, sensitivity and likelihood ratio. The cut-off point for each analyte was chosen based on the highest likelihood ratio in the sensitivity vs. 1-specificity plot, favoring a higher sensitivity than specificity values, to obtain assays with higher likelihood of reliability for each analyte. Positive, and negative predictive values were also calculated along with overall assay accuracy. These data are presented in Tables 8-13, below.

TABLE 8
Demographics and injury characteristics of pediatric traumatic brain
injury subjects with favorable and unfavorable neurological outcomes

| | | | Favorable | | Unfavorable | | |
|---|---|---|---|---|---|---|---|
| | | | Outcome | | Outcome | | |
| | Control | | (GOS-E Peds >4) | | (GOS-E Peds ≤4) | | |
| Characteristic | (n = 19) | | (n = 10) | | (n = 10) | | p-value |
| Age in years, mean (SD) ^{a} | 10.58 | (5.76) | 3.9 | (3.63) | 5.7 | (6.25) | 0.006 |
| Sex, n (%) ^{a} | | | | | | | 0.187 |
| Female | 10 | (53) | 3 | (30) | 2 | (20) | |
| Male | 9 | (47) | 7 | (70) | 8 | (80) | |
| Race, n(%) ^{a} | | | | | | | 0.212 |
| White | 9 | (47) | 6 | (60) | 4 | (40) | |
| African American | 6 | (32) | 2 | (20) | 4 | (40) | |
| Unknown | 4 | (21) | 2 | (20) | 2 | (20) | |
| Payer status, n(%) ^{a} | | | | | | | 0.621 |
| Medicaid | 6 | (32) | 4 | (40) | 5 | (50) | |
| Other | 13 | (68) | 6 | (60) | 5 | (50) | |
| GCS, n(%) ^{b} | | | | | | | 0.054 |
| Severe (GCS: 3-8) | 0 | | 1 | (10) | 6 | (60) | |
| Moderate (GCS: 9-12) | 0 | | 1 | (10) | 1 | (10) | |
| Mild (GCS: 13-15) | 0 | | 8 | (80) | 3 | (30) | |
| ISS Score, n(%) ^{b} | | | | | | | 0.132 |
| Minor (1-8) | | | 2 | (20) | 0 | | |
| Moderate (9-15) | | | 1 | (10) | 0 | | |
| Serious (16-24) | NA | | 0 | | 1 | (10) | |
| Severe (25-49) | | | 6 | (60) | 4 | (40) | |
| Critical (75) | | | 1 | (10) | 5 | (50) | |
| BMI, mean (SD) ^{a} | 21.32 | (6.79) | 16.02 | (2.41) | 30.95 | (30.99) | 0.236 |

^{a} P values demonstrate comparison of frequency distributions by control, favorable, and unfavorable outcomes
^{b} P values for comparison of frequency distributions by favorable, and unfavorable outcomes
Glasgow Outcome Scale Extended Pediatrics (GOS-E Peds), Glasgow Coma Scale (GCS), Injury Severity Score (ISS), Body Mass Index (BMI).

TABLE 9
ROC Analysis HC vs TBI

| Biomarker | Area | Std. Error | 95% C.I. | p-value |
|---|---|---|---|---|
| Caspase-1 at 0 h | 0.599 | 0.07956 | 0.4437 to 0.7556 | 0.2407 |
| Caspase-1 at 24 h | 0.5820 | 0.09800 | 0.3899 to 0.7741 | 0.3825 |
| Caspase-1 at 48 h | 0.6365 | 0.08718 | 0.4656 to 0.8073 | 0.1377 |
| ASC at 0 h | 0.7530 | 0.07050 | 0.6148 to 0.8912 | 0.0041 |
| ASC at 24 h | 0.6931 | 0.08486 | 0.5268 to 0.8594 | 0.0397 |
| ASC at 48 h | 0.5965 | 0.09479 | 0.4107 to 0.7823 | 0.3160 |
| IL-18 at 0 h | 0.6065 | 0.07807 | 0.4535 to 0.7595 | 0.1784 |
| IL-18 at 24 h | 0.5247 | 0.08724 | 0.3537 to 0.6957 | 0.7727 |
| IL-18 at 48 h | 0.5663 | 0.08890 | 0.3920 to 0.7405 | 0.4521 |
| IL-1β at 0 h | 1 | 0 | 1 to 1 | <0.0001 |
| IL-1β at 24 h | 0.6096 | 0.1104 | 0.3933 to 0.8259 | 0.3349 |
| IL-1β at 48 h | 0.6942 | 0.09116 | 0.5155 to 0.8729 | 0.0727 |

TABLE 10
Cut-off point in serum of TBI patients

| | Cut-off | | | | | | |
|---|---|---|---|---|---|---|---|
| | point | Sensitivity | Specificity | | PPV | NPV | Accuracy |
| Biomarker | (pg/ml) | (%) | (%) | LR | (%) | (%) | (%) |
| Caspase-1 at 0 h | >3.440 | 71% | 50% | 1.412 | 73% | 48% | 64% |
| Caspase-1 at 24 h | >2.845 | 81% | 44% | 1.457 | 63% | 67% | 64% |
| Caspase-1 at 48 h | <4.345 | 74% | 39% | 1.209 | 61% | 54% | 59% |
| ASC at 0 h | >513.5 | 71% | 61% | 1.837 | 74% | 58% | 67% |
| ASC at 24 h | >479 | 76% | 56% | 1.714 | 67% | 67% | 67% |
| ASC at 48 h | >429 | 74% | 44% | 1.83723 | 58% | 62% | 59% |
| IL-18 at 0 h | <256.0 | 76% | 48% | 1.457 | 45% | 78% | 58% |
| IL-18 at 24 h | >167.5 | 77% | 38% | 1.243 | 50% | 67% | 55% |
| IL-18 at 48 h | >178.5 | 74% | 43% | 1.293 | 54% | 64% | 58% |
| IL-1β at 0 h | >1.090 | 100% | 91% | 11 | 76% | 100% | 93% |
| IL-1β at 24 h | >0.4830 | 71% | 45% | 11 | 46% | 71% | 55% |
| IL-1β at 48 h | >1.090 | 78% | 55% | 11 | 46% | 83% | 62% |

TABLE 11
ROC analysis of inflammatory biomarkers
as predictors of TBI severity

| Biomarker | Area | Std. Error | 95% C.I. | p-value |
|---|---|---|---|---|
| Caspase-1 | 0.5 | 0.1075 | 0.2893 to 0.7107 | >0.9999 |
| ASC | 0.7204 | 0.09029 | 0.5434 to 0.8973 | 0.0315 |
| IL-18 | 0.5223 | 0.09638 | 0.3334 to 0.7112 | 0.8181 |
| IL-1β | 0.6303 | 0.1142 | 0.4064 to 0.8542 | 0.2645 |

TABLE 12
Cut-off point in serum of TBI patients

| | Cut-off | | | | | | |
|---|---|---|---|---|---|---|---|
| | point | Sensitivity | Specificity | | PPV | NPV | Accuracy |
| Biomarker | (pg/ml) | (%) | (%) | LR | (%) | (%) | (%) |
| Caspase-1 | >2.51 | 63% | 33% | 0.9375 | 45% | 50% | 47% |
| ASC | >469.5 | 73% | 55% | 1.65 | 58% | 71% | 63% |
| IL-18 | <256 | 69% | 48% | 1.31 | 50% | 67% | 57% |
| IL-1β | <0.57 | 67% | 45% | 1.22 | 62% | 45% | 58% |

TABLE 13
ROC analysis of inflammatory biomarkers as predictors of Outcome

| Biomarker | Area | Std. Error | 95% C.I. | p-value |
|---|---|---|---|---|
| IL-18 at 0 h (12 month average) | 0.7067 | 0.1629 | 0.3874 to 1 | 0.1761 |
| IL-18 at 48 h (12 month average) | 0.8222 | 0.1439 | 0.5401 to 1 | 0.0532 |
| IL-1β at 0 h (12 month average) | 0.8308 | 0.1003 | 0.6342 to 1 | 0.0341 |

The above disclosure and example generally describe the present invention and is provided for purposes of illustration and is not intended to limit the scope of the invention. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

All publications, patents and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize equivalents to the specific embodiments of the invention described herein. Such equivalents are intended to be encompassed by the following claims.

1. A method for assessing and treating Traumatic Brain Injury (TBI) in a patient, said method comprising
providing a biological or tissue sample from the patient;
determining an amount of one or more inflammatory cytokine in the patient biological or tissue sample;
determining whether said inflammatory protein meets a pre-determined cut-off value for said inflammatory cytokine, and
administering to the patient a composition which inactivates or neutralizes the amount of inflammatory cytokine such that the amount of inflammatory cytokine is increased or decreased toward a predetermined normal reference value.

2. The method of claim 1, wherein the composition comprises a small molecule drug or large molecule antibody or antibody fragment active against the inflammatory cytokine such that the inflammatory cytokine is inactivated or neutralized.

3. The method of claim 1, wherein the patient is an adult.

4. The method of claim 1, wherein the patient is a pediatric patient.

5. The method of claim 1, wherein the inflammatory cytokine is selected from the group consisting of caspase-1, ASC, IL-18, TNF-α, IL-6, IL-4, IL-10, IL-8, IL-2, and IL-10.

6. The method of claim 3, wherein the cut-off value of said inflammatory cytokine is ±20% of:

| Caspase-1 | >0.8150 | pg/ml |
|---|---|---|
| ASC | >284 | pg/ml |
| IL-18 | >156 | pg/ml |
| TNF-α | >2.202 | pg/ml |
| IL-6 | >6.443 | pg/ml |
| IL-4 | >0.03868 | |
| IL-10 | >0.6527 | pg/ml |
| IL-8 | >29.18 | pg/ml |
| IL-2 | <0.5145 | pg/ml. |

7. The method of claim 4, wherein the cut-off value of said inflammatory cytokine is ±20% of:

| | | |
|---|---|---|
| Caspase-1 | >2.51 | pg/ml |
| ASC | >469.5 | pg/ml |
| IL-18 | <256 | pg/ml |
| IL-1β | <0.57 | pg/ml. |

8. The method of claim 1, wherein the administered composition comprises one or more small molecule selected from the group consisting of methylprednisolone, 17α-estradiol, 170-estradiol, ginsenoside, progesterone, simvastatin, deprenyl, minocycline, resveratrol, glutamate receptor antagonist or NMDA receptor antagonist, and an antioxidant.

9. The method of claim 1, wherein the administered composition comprises one or more antibody or antibody fragment selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; or SEQ ID NO:41.

10. The method of claim 1, wherein the administered composition comprises one or more small molecule selected from the group consisting of methylprednisolone, 17α-estradiol, 170-estradiol, ginsenoside, progesterone, simvastatin, deprenyl, minocycline, resveratrol, and other glutamate receptor antagonists (e.g., NMDA receptor antagonists) and an antioxidant and one or more antibody or antibody fragment selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; or SEQ ID NO:41.

11. The method of claim 1, wherein said biological sample is cerebrospinal fluid (CSF), CNS microdialysate, saliva, serum, plasma, or urine.

12. The method of claim 1, further comprising: measuring the level of said at least one inflammatory cytokine in a biological sample obtained from the patient following neuroprotective treatment; preparing a treatment protein signature associated with a positive response to the neuroprotective treatment, wherein the treatment protein signature comprises a reduced level of at least one inflammatory cytokine; and identifying patients exhibiting the presence of the treatment protein signature as responding positively to the neuroprotective treatment.

13. A kit for preparing an inflammatory cytokine profile associated with TBI comprising a labeled-binding partner that specifically binds to one or more inflammasome proteins, wherein said one or more inflammasome proteins are selected from the group consisting of caspase-1, ASC, IL-18, TNF-α, IL-6, IL-4, IL-10, IL-8, IL-2, and IL-10, and combinations thereof.

14. The kit of claim 13, wherein the kit comprises labeled antibody or a fragment thereof which binds specifically to each of caspase-1, ASC, IL-18, TNF-α, IL-6, IL-4, IL-10, IL-8, IL-2, and IL-10.

15. The kit of claim 13, wherein the labeled antibody or fragment thereof, aptamer, or peptide.

16. A method of determining a prognosis for a patient with traumatic brain injury (TBI) comprising:
providing a biological sample obtained from the patient within a week of injury; and
measuring the level of at least one inflammasome protein in the biological sample to prepare an inflammatory cytokine profile, wherein the level of inflammatory cytokine outside the cut-off value (±20%) for the inflammatory cytokine is indicative of the prognosis of the patient.

17. The method of claim 16, wherein said at least one inflammatory cytokine is caspase-1, ASC, IL-18, TNF-α, IL-6, IL-4, IL-10, IL-8, IL-2, and IL-10, or combinations thereof.

18. The method of claim 16, wherein said biological sample is CSF, CNS microdialysate, saliva, serum, plasma, or urine.

19. The method of claim 16, wherein the patient is a pediatric patient.


SEQUENCE LISTING (41 sequences)

SEQ ID NO: 1 - protein, 16 aa, Rattus norvegicus
ALRQTQPYLVTDLEQS

SEQ ID NO: 2 - protein, 14 aa, Homo sapiens
RESQSYLVEDLERS

SEQ ID NO: 3 - protein, 19 aa, Homo sapiens
CEYYTEIREREREKSEKGR

SEQ ID NO: 4 - protein, 15 aa, Rattus norvegicus
MEESQSKEESNTEGC

SEQ ID NO: 5 - protein, 21 aa, Homo sapiens
KKFKLKLLSVPLREGYGRIPR

SEQ ID NO: 6 - protein, 7 aa, synthetic construct
  REGION 1..7: note = Heavy Chain (H) CDR1
TSGMGVS

SEQ ID NO: 7 - protein, 16 aa, synthetic construct
  REGION 1..16: note = Heavy Chain (H) CDR2
HIYWDDDKRYNPSLKS

SEQ ID NO: 8 - protein, 11 aa, synthetic construct
  REGION 1..11: note = Heavy Chain (H) CDR3
STPIVANAMDY

SEQ ID NO: 9 - DNA, 21 bp, synthetic construct
  misc_feature 1..21: note = Heavy Chain( H) CDR1
  source 1..21: mol_type = other DNA
actagtggaatgggtgtgagc

SEQ ID NO: 10 - DNA, 48 bp, synthetic construct
  misc_feature 1..48: note = Heavy Chain (H) CDR2
  source 1..48: mol_type = other DNA
cacatttattgggatgatgataagcgctacaacccatctctgaagagc

SEQ ID NO: 11 - DNA, 33 bp, synthetic construct
  misc_feature 1..33: note = Heavy Chain (H) CDR3
  source 1..33: mol_type = other DNA
agcacccccatcgtggccaacgccatggactac

SEQ ID NO: 12 - protein, 15 aa, synthetic construct
  REGION 1..15: note = Light (Kappa) (L) Chain CDR1
KASQSVDYDGDSYMN

SEQ ID NO: 13 - protein, 7 aa, synthetic construct
  REGION 1..7: note = Light (Kappa) (L) Chain CDR2
AASNLES

SEQ ID NO: 14 - protein, 9 aa, synthetic construct
  REGION 1..9: note = Light (Kappa) (L) Chain CDR3
QQSNEDPYT

SEQ ID NO: 15 - DNA, 45 bp, synthetic construct
  misc_feature 1..45: note = Light (Kappa) (L) Chain CDR1
  source 1..45: mol_type = other DNA
aaggccagccagagtgttgactacgacggcgacagttacatgaat

SEQ ID NO: 16 - DNA, 21 bp, synthetic construct
  misc_feature 1..21: note = Light (Kappa) (L) Chain CDR2
  source 1..21: mol_type = other DNA
gccgcatctaacctggaatcc

SEQ ID NO: 17 - DNA, 27 bp, synthetic construct
  misc_feature 1..27: note = Light (Kappa) (L) Chain CDR3
  source 1..27: mol_type = other DNA
cagcaatctaatgaggacccttacact

SEQ ID NO: 18 - protein, 121 aa, synthetic construct
  REGION 1..121: note = Variable Heavy (VH) 1 Chain
QVTLKESGPAIVKPTQTLTLTCSFSGFSLSTSGMGVSWIRQPSGKGLEWLAHIYWDDDKRYNPSLKSRLTISKDSSKNQVVLKITSVDPVDTATYSCARSTPIVANAMDYWGQGTSVTVSS

SEQ ID NO: 19 - protein, 121 aa, synthetic construct
  REGION 1..121: note = Variable Heavy (VH) 2 Chain
QVTLKESGPALVKPTQTLTLTCSFSGFSLSTSGMGVSWIRQPAGKGLEWLAHIYWDDDKRYNPSLKSRLTISKDSSKNQVVLTMTNMDPVDTATYSCARSTPIVANAMDYWGQGTLVTVSS

SEQ ID NO: 20 - protein, 121 aa, synthetic construct
  REGION 1..121: note = Variable Heavy (VH) 3 Chain
QVTLKESGPALVKPTQTLTLTCSFSGFSLSTSGMGVSWIRQPAGKGLEWLAHIYWDDDKRYNPSLKSRLTISKDSSKNQVVLTMTNMDPVDTATYYCARSTPIVANAMDYWGQGTLVTVSS

SEQ ID NO: 21 - protein, 121 aa, synthetic construct
  REGION 1..121: note = Variable Heavy (VH) 4 Chain
QVTLKESGPALVKPTQTLTLTCTFSGFSLSTSGMGVSWIRQPAGKGLEWLAHIYWDDDKRYNPSLKSRLTISKDTSKNQVVLTMTNMDPVDTATYYCARSTPIVANAMDYWGQGTLVTVSS

SEQ ID NO: 22 - protein, 121 aa, synthetic construct
  REGION 1..121: note = Variable Heavy (VH) Chimeric (O) Chain
QVTLKESGPGILQPSQTLSLTCSFSGFSLSTSGMGVSWIRQPSGKGLEWLAHIYWDDDKRYNPSLKSRLTISKDSSSNQVFLKITSVDTADTATYSCARSTPIVANAMDYWGQGTSVTVSS

SEQ ID NO: 23 - DNA, 363 bp, synthetic construct
  misc_feature 1..363: note = Variable Heavy (VH) 1 Chain
  source 1..363: mol_type = other DNA
caggtcaccttgaaggagtctggtcctgccatcgtgaaacccacacagaccctcacgctgacctgcagcttctctgggttctcactcagcactagtggaatgggtgtgagctggatccgtcagccctcaggaaagggcctggagtggcttgcacacatttattgggatgatgataagcgctacaacccatctctgaagagcaggctcaccatctccaaggacagctccaaaaaccaggtggtccttaaaatcaccagcgtggaccctgtggacacagccacatattcctgtgcacggagcacccccatcgtggccaacgccatggactactggggccaaggaaccagcgtcaccgtctcctca

SEQ ID NO: 24 - DNA, 363 bp, synthetic construct
  misc_feature 1..363: note = Variable Heavy (VH) 2 Chain
  source 1..363: mol_type = other DNA
caggtcaccttgaaggagtctggtcctgccctggtgaaacccacacagaccctcacgctgacctgcagcttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacagctccaaaaaccaggtggtccttacaatgaccaacatggaccctgtggacacagccacatattcctgtgcacggagcacccccatcgtggccaacgccatggactactggggccaaggaaccctggtcaccgtctcctca

SEQ ID NO: 25 - DNA, 363 bp, synthetic construct
  misc_feature 1..363: note = Variable Heavy (VH) 3 Chain
  source 1..363: mol_type = other DNA
caggtcaccttgaaggagtctggtcctgccctggtgaaacccacacagaccctcacgctgacctgcagcttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacagctccaaaaaccaggtggtccttacaatgaccaacatggaccctgtggacacagccacatattactgtgcacggagcacccccatcgtggccaacgccatggactactggggccaaggaaccctggtcaccgtctcctca

SEQ ID NO: 26 - DNA, 363 bp, synthetic construct
  misc_feature 1..363: note = Variable Heavy (VH) 4 Chain
  source 1..363: mol_type = other DNA
caggtcaccttgaaggagtctggtcctgccctggtgaaacccacacagaccctcacgctgacctgcaccttctctgggttctcactcagcactagtggaatgggtgtgagctggatccgtcagcccgccggaaagggcctggagtggcttgcacacatttattgggatgatgataagcgctacaacccatctctgaagagcaggctcaccatctccaaggacacctccaaaaaccaggtggtccttacaatgaccaacatggaccctgtggacacagccacatattactgtgcacggagcacccccatcgtggccaacgccatggactactggggccaaggaaccctggtcaccgtctcctca

SEQ ID NO: 27 - DNA, 363 bp, synthetic construct
  misc_feature 1..363: note = Variable Heavy (VH) Chimeric (O) Chain
  source 1..363: mol_type = other DNA
caggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggattcctccagcaaccaggtcttcctcaagatcaccagtgtggacactgcagatactgccacatactcctgtgctcgaagtactccgattgtagctaatgctatggactactggggtcaaggaacctcagtcaccgtctcctca

SEQ ID NO: 28 - protein, 111 aa, synthetic construct
  REGION 1..111: note = Variable Kappa Light (VL) 1 Chain
DIVLTQSPDSLAVSLGERATINCKASQSVDYDGDSYMNWYQQKPGQPPKLLIYAASNLESGIPARFSGSGSGTDFTLTISSLQEEDVATYYCQQSNEDPYTFGQGTKLEIK

SEQ ID NO: 29 - protein, 111 aa, synthetic construct
  REGION 1..111: note = Variable Kappa Light (VL) 2 Chain
DIVLTQSPDSLAVSLGERATINCKASQSVDYDGDSYMNWYQQKPGQPPKLLIYAASNLESGIPARFSGSGSGTDFTLTISSLQPEDVATYYCQQSNEDPYTFGQGTKLEIK

SEQ ID NO: 30 - protein, 111 aa, synthetic construct
  REGION 1..111: note = Variable Kappa Light (VL) 3 Chain
DIVMTQSPDSLAVSLGERATINCKASQSVDYDGDSYMNWYQQKPGQPPKLLIYAASNLESGIPARFSGSGSGTDFTLTISSLQPEDVATYYCQQSNEDPYTFGQGTKLEIK

SEQ ID NO: 31 - protein, 111 aa, synthetic construct
  REGION 1..111: note = Variable Kappa Light (VL) Chimeric (O) Chain
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 32 - DNA, 333 bp, synthetic construct
  misc_feature 1..333: note = Variable Kappa Light (VL) 1 Chain
  source 1..333: mol_type = other DNA
gacatcgtgctgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcaggaggaagatgtggcaacttattactgtcagcaatctaatgaggacccttacacttttggccaggggaccaagctggagatcaaa

SEQ ID NO: 33 - DNA, 333 bp, synthetic construct
  misc_feature 1..333: note = Variable Kappa Light (VL) 2 Chain
  source 1..333: mol_type = other DNA
gacatcgtgctgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcagcctgaagatgtggcaacttattactgtcagcaatctaatgaggacccttacacttttggccaggggaccaagctggagatcaaa

SEQ ID NO: 34 - DNA, 333 bp, synthetic construct
  misc_feature 1..333: note = Variable Kappa Light (VL) 3 Chain
  source 1..333: mol_type = other DNA
gacatcgtgatgacccagtctccagactccctggctgtgtctctgggcgagagggccaccatcaactgcaaggccagccagagtgttgactacgacggcgacagttacatgaattggtaccagcagaaaccaggacagcctcctaagctgctcatttacgccgcatctaacctggaatccggcatccctgcccgattcagtggcagcgggtctgggacagatttcactctcaccatcagcagcctgcagcctgaagatgtggcaacttattactgtcagcaatctaatgaggacccttacacttttggccaggggaccaagctggagatcaaa

SEQ ID NO: 35 - DNA, 333 bp, synthetic construct
  misc_feature 1..333: note = Variable Kappa Light (VL) Chimeric (O) Chain
  source 1..333: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctggcatcccagccaggtttagtggcagtgggtctgggacagacttcaccctcaacatccatcctgtggaggaggaggatgctgcaacctattactgtcagcaaagtaatgaggacccgtacacgttcggaggggggaccaagctggaaataaaa

SEQ ID NO: 36 - protein, 753 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 37 - protein, 670 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQK

SEQ ID NO: 38 - protein, 654 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKM

SEQ ID NO: 39 - protein, 42 aa, Homo sapiens [sequence omitted]

SEQ ID NO: 40 - protein, 40 aa, Homo sapiens
DAEFRHDSGYEVHHQKLVFFAEDVGSNKGAIIGLMVGGVV

SEQ ID NO: 41 - protein, 542 aa, Homo sapiens
SSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD